(19) United States
(12) Patent Application Publication
(10) Pub. No.: US 2008/0248584 A1
(43) Pub. Date: Oct. 9, 2008

(54) Trityl derivatives for enhancing mass spectrometry

(76) Inventors: Mikhail Sergeevich SHCHEPINOV, Oxford (GB); Edwin Mellor SOUTHERN, Oxford (GB)

Correspondence Address: WENDEROTH, LIND & PONACK, L.L.P., 2033 K STREET N. W., SUITE 800, WASHINGTON, DC, 20006-1021, US

(21) Appl. No.: 11/892,372

(22) Filed: Aug. 22, 2007

Related U.S. Application Data
(63) Continuation of application No. 10/581,682, now abandoned.

(30) Foreign Application Priority Data
Dec. 8, 2003 (GB) 0328414.8
Sep. 22, 2004 (EP) 04104605.3
Sep. 22, 2004 (GB) 0421110.8

(51) Int. Cl. G01N 33/68; C08F 251/00; C07K 1/107; C07D 207/00; C07C 215/00; G01N 33/50; C07C 69/00; C07C 233/00; C07D 235/02

(52) U.S. Cl. 436/86; 527/300; 527/200; 548/542; 564/443; 560/57; 564/172; 548/303.7; 436/94

(57) ABSTRACT

Compounds of formula (IIa):

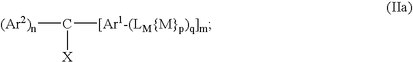

are provided where:
X is a group capable of being cleaved from the α-carbon atom to form an ion of formula (I′)

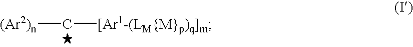

C [symbol] is a carbon atom bearing a single positive charge or a single negative charge;

The invention further provides compounds of formula (IIb):

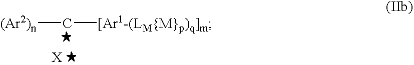

where:
X [symbol] is a counter-ion to C [symbol] .

The compounds of formula (IIa) and (IIb) may form ions of formula (I′) by either cleaving the C—X bond between X and the α-carbon atoms in the case of the compounds of formula (IIa) or dissociating X [symbol] in the case of compounds of formula (IIb).

### TECHNICAL FIELD

This invention relates to derivatised biopolymers and ions obtainable therefrom. The invention further relates to compounds and solid supports useful for producing the derivatised biopolymers and ions of the invention.

### BACKGROUND OF THE INVENTION

Mass spectrometry is a versatile analytical technique possessing excellent detection range and speed of detection with respect to High Performance Liquid Chromatography (HPLC), Gas Chromatography (GC), Infra-Red (IR) and Nuclear Magnetic Resonance (NMR).

However, many biopolymers, such as carbohydrates and proteins, are difficult to analyse using mass spectrometry due to significant difficulties in ionising the biopolymer, even using Matrix Assisted Laser Desorption/Ionisation Time Of Flight (MALDI-TOF) techniques. Despite the considerable resolving power of 2D-PAGE, this technology has fallen far short of the ultimate goal of displaying the whole proteome in a single experiment, as many proteins are resistance to 2D-PAGE analysis (e.g. those with low or high molecular masses, membrane proteins, proteins with extreme isoelectric points, etc.). Many proteins are thus invisible to 2-D PAGE [Cravatt & Sorensen (2000) Current Opinion in Chemical Biology vol. 4, p. 663-668].

There is thus a need for improvements in mass spectrometry analysis of biopolymers.

### DISCLOSURE OF THE INVENTION

It has now been found that covalent attachment of trityl derivatives to biopolymers can improve the ionisation properties of the biopolymer. The ions (formula (I) below) formed by ionisation of the derivatised biopolymers are particularly suitable for mass spectrometry analysis, and biopolymers derivatised as specified in formulae (IIIa) and (IIIb) below can be readily ionised.

Whereas triphenylmethyl derivatives covalently attached to certain biopolymers (e.g. DNA) are known in the prior art [e.g. Chem. Soc. Rev. (2003) 32, p. 3-13], the prior art attaches the polymer to the x-triphenylmethyl carbon atom through a non-aromatic linker. In contrast, under the present invention the biopolymer is attached to the α-triarylmethyl carbon atom via an aromatic group adjacent to the central carbon atom. Consequently, ionisation of the prior art derivatives results in separation of the triphenylmethyl derivative and the biopolymer, whereas according to the present invention the biopolymer remains bound to the trityl derivative on ionisation, thereby allowing analysis of the biopolymer by mass spectrometry.

The invention provides methods of forming ions from covalent or ionic compounds and solid substrates.

### Derivatised Biopolymers

The invention provides a method of forming an ion of formula (I):

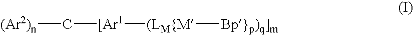

comprising the steps of:
(i) reacting a compound of the formula (IIa):

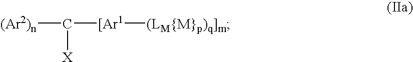

with a biopolymer, B_{P}, having at least one group capable of reacting with M to form a covalent linkage, to provide a biopolymer derivative of the formula (IIIa):

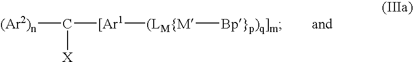

(ii) cleaving the C—X bond between X and the α-carbon atom of the derivative of formula (IIIa) to form the ion of formula (I);
   where:
C [symbol] is a carbon atom bearing a single positive charge or a single negative charge;
X is a group capable of being cleaved from the α-carbon atom to form an ion of formula (I);
M is independently a group capable of reacting with B_{P} to form the covalent linkage;
B_{P}′ is independently the biopolymer residue of B_{P} produced on formation of the covalent linkage;
M′ is independently the residue of M produced on formation of the covalent linkage;
Ar¹ is independently an aromatic group or an aromatic group substituted with one or more A;
Ar² is independently an aromatic group or an aromatic group substituted with one or more A;
   optionally wherein (a) two or three of the groups Ar¹ and Ar² are linked together by one or more L⁵, where L⁵ is independently a single bond or a linker atom or group; and/or (b) two or three of the groups Ar¹ and Ar² together form an aromatic group or an aromatic group substituted with one or more A;
A is independently a substituent;
L_{M} is independently a single bond or a linker atom or group;
n=0, 1 or 2 and m=1, 2, or 3, provided the sum of n+m=3;
p independently=1 or more; and
q independently=1 or more.

The invention further provides a method of forming an ion of formula (I), comprising the steps of:
(i) reacting a compound of the formula (IIb):

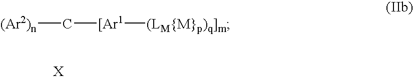

with a biopolymer, B_{P}, having at least one group capable of reacting with M to form a covalent linkage, to provide a biopolymer derivative of the formula (IIIb):

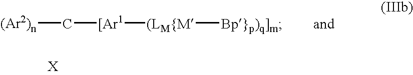

dissociating X [symbol] from the derivative of formula (IIIb), to form the ion of formula (I);
where:
X [symbol] is a counter-ion to C [symbol] ;
and C [symbol] M, B_{P}′, M′, Ar¹, Ar², L_{M}, n, m, p and q are as defined above.

The compounds of formulae (IIa) or (IIb) may optionally be purified after step (i).

The invention also provides biopolymer derivatives of the formula (IIIa) or (IIb), as defined above. The biopolymer derivatives of the invention have enhanced ionisability with respect to free biopolymer, B_{P}. Advantageously, the biopolymer derivatives may not require a matrix (e.g. as used in MALDI-MS) in order to elicit ionisation, although a matrix may help to enhance ionisation. Preferably, ionisation may be obtained without requiring acid treatment, in particular by direct laser illumination.

The invention also provides ions of formula (I), as defined above. These ions are stabilised by the resonance effect of the aromatic groups Ar¹ and Ar². Electron-withdrawing groups, when C [symbol] is an anion, or electron-donating groups, when C [symbol] is a cation, may optionally be provided on Ar¹ and/or Ar² to assist this resonance effect. Consequently, the biopolymer derivatives of the invention readily form ions of formula (I) relative to the native biopolymer, B_{P}.

The ions of formula (I) are generally only ever seen on a mass spectrum with a single charge, which is advantageous since it reduces cluttering of the mass spectrum.

The invention also provides compounds of the formula (IIa) and (IIb), as defined above. As mentioned above, these compounds are useful for forming ions of formula (I). As the difference in the molecular mass of the ions of formula (I) and that of the free biopolymer can be accurately calculated, the derivatised compounds of the invention allow analysis of the biopolymer B_{P}, which may be otherwise difficult or impossible to analyse using known mass spectrometrical techniques.

Other advantageous features of the compounds of the invention include more uniformity of the signal intensity between different analytes (useful for quantitative studies) and similar desorption properties between compounds with different, but close, masses, so that techniques such as isotope coded affinity tagging (ICAT) can be employed with the compounds of the invention.

The homogeneous methods of the invention are particularly appropriate for small molecules, e.g. amines.

### Solid Supports

The ions of formula (I) may also be formed using a derivatised solid support.

The invention therefore provides a method of forming an ion of formula (I) comprising the steps of:
(i) reacting a solid support of formula (IVai), (IVaii), or (IVaiii):

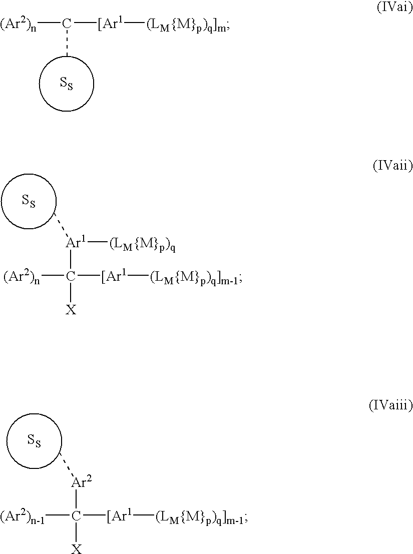

with a biopolymer, B_{P}, having at least one group capable of reacting with M to form a covalent linkage, to provide a modified solid support of the formula (Vai), (Vaii), or (Vaiii), respectively:

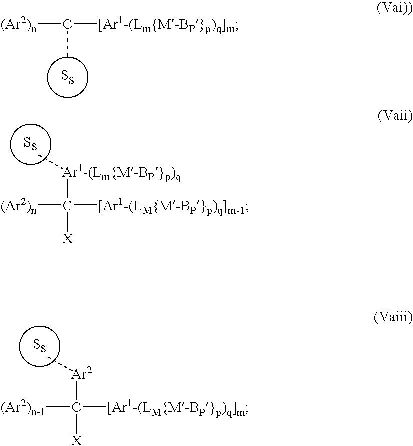

and either:
(iia) for modified solid supports of formula (Vai) cleaving the C—S_{S} bond between the α-carbon atom of the modified solid support of formula (Vai) and the solid support S_{S} to form the ion of formula (I);
(iib) for modified solid supports of formula (Vaii), either simultaneously or sequentially, cleaving the C—X bond between X and the α-carbon atom and cleaving the S_{S}—Ar¹ bond between the solid support and the Ar¹ group to form the ion of formula (I); or
(iic) for modified solid supports of formula (Vaiii), either simultaneously or sequentially, cleaving the C—X bond between X and the α-carbon atom and cleaving the S_{S}—Ar² bond between the solid support and the Ar² group to form the ion of formula (I); where:
X, Ar¹, Ar², B_{P}′, L_{M}, M, M′, n, m, p and q are as defined above;
S_{S} is a solid support;
C—S_{S} comprises a cleavable bond between C and S_{S};
S_{S}—Ar¹ comprises a cleavable bond between Ar¹ and S_{S}; and

S_{S}—Ar² comprises a cleavable bond between Ar² and S_{S}.

The cleavable bond of C—S_{S}, S_{S}—Ar¹ or S_{S}—Ar² may be a covalent, ionic, hydrogen, dipole-dipole or van der Waals bond.

The invention further provides a method of forming an ion of formula (I) comprising the steps of:
(i) reacting a solid support of formula (IVbii) or (IVbiii):

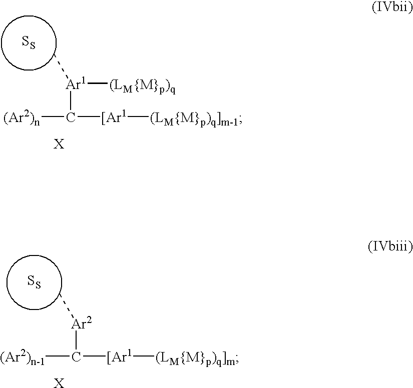

with a biopolymer, B_{P}, having at least one group capable of reacting with M to form a covalent linkage, to provide a modified solid support of the formula (Vbii) or (Vbiii), respectively:

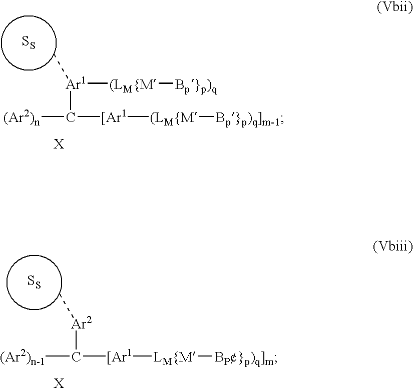

and either:
(iia) for modified solid supports of formula (Vbii), either simultaneously or sequentially, dissociating X [symbol] from the derivative of formula (Vbii) and cleaving the S_{S}—Ar¹ bond between the solid support and the Ar¹ group to form an ion of formula (I); or
(iib) for modified solid supports of formula (Vbiii), either simultaneously or sequentially, dissociating X [symbol] from the derivative of formula (Vbiii) and cleaving the S_{S}—Ar² bond between the solid support and the Ar² group to form an ion of formula (I);
   where: X [symbol] Ar¹, Ar², B_{P}′, L_{M}, M, M′, n, m, p, q, S_{S}, C—S_{S}, S_{S}—Ar¹ and S_{S}—Ar² are as defined above.

The invention further provides a method of forming an ion of formula (I) comprising the steps of:
(i) reacting a solid support of formula (IVaiv) or (IVbiv):

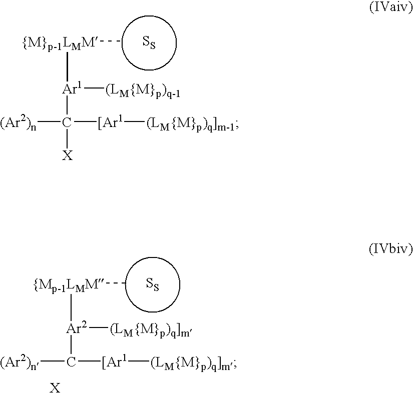

with a biopolymer, B_{P}, having at least one group capable of reacting with M to form a covalent linkage, to provide a modified solid support of the formula (Vaiv) or (Vbiv), respectively:

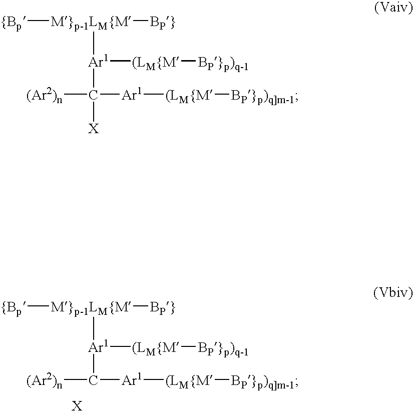

and either:
(iia) for modified solid supports of formula (Vaiv), cleaving the C—X bond between X and the α-carbon atom to form the ion of formula (I); or
(iib) for modified solid supports of formula (Vbiv), dissociating X [symbol] from the derivative of formula (Vbiv) to form the ion of formula (I);
   where:
X, X [symbol] Ar¹, Ar², B_{P}′, L_{M}, M, M′, p, q, n, m, and S_{S} are as defined above;
M″---S_{S} comprises a bond between M″ and S_{S}; and
M″ is the same as M except that S_{S} is bound to a portion of M which does not form part of M′.

In this embodiment of the invention, the solid support is bound to a part of group M″ which does not go on to form the residue M′. Thus, the derivatised biopolymer will be released from the solid support during the derivativisation step and an additional step of cleaving the biopolymer from the solid support is not required.

The modified solid supports of formulae (Vai), (Vaii), (Vaiii), (Vaiv), (Vbii), (Vbiii) or (Vbiv) may optionally be washed after step (i).

The invention also provides solid supports of the formulae (IVai), (IVaii), (IVaiii), (IVaiv), (IVbii), (IVbiii) and (IVbiv), as defined above. Similarly, the invention provides modified solid supports of the formulae (Vai), (Vaii), (Vaiii), (Vaiv), (Vbii), (Vbiii), and (Vbiv), as defined above.

The heterogeneous methods of the invention are particularly appropriate for synthetic biopolymers, e.g. oligonucleotides, peptides and carbohydrates.

### Methods of Analysis

The invention also provides a method for analysing a biopolymer, B_{P}, comprising the steps of:
(i) reacting the biopolymer B_{P} with a compound of formula (IIa) or (IIb) or a solid support of formula (IVai), (IVaii), (IVaiii), (IVaiv), (IVbii), (IVbiii) or (IVbiv);
(ii) providing an ion of formula (I); and
(iii) analysing the ion of formula (I) by mass spectrometry.

The biopolymer will typically have been obtained using a preparative or analytical process. For example, it may have been purified using various separation methods (e.g. 1-dimensional or 2-dimensional, reverse-phase or normal-phase separation, by e.g. chromatography or electrophoresis) and the separation may be based on any of a number of characteristics (e.g. isoelectric point, molecular weight, charge, hydrophobicity, etc.). Typical methods include 2D SDS-PAGE, 2D liquid chromatography (e.g. Multidimensional Protein Identification Technology, MudPIT, or 2D HPLC methods). The separation method can preferably interface directly with the mass spectrometer.

Known analytical techniques can thus be adapted or improved by the method of the invention. A particularly preferred method involves 2D-PAGE of a biopolymer, or mixture of biopolymers, selection of a spot of interest in the electrophoretogram, and then derivatisation and analysis of that spot using the techniques of the invention. The biopolymer may be proteolytically digested prior to its analysis (typically within the PAGE gel, but optionally digested after extraction from the gel) and/or may itself be the product of a proteolytic digest.

The invention also provides, in a method for analysing a biopolymer, B_{P}, the improvement consisting of: (i) reacting a biopolymer, B_{P} with a compound of formula (IIa) or (IIb) or a solid support of formula (IVai), (IVaii), (IVaiii), (IVaiv), (IVbii), (IVbiii) or (IVbiv); (ii) providing an ion of formula (I); and (iii) analysing the ion by mass spectrometry.

Typically, the analysis by mass spectrometry is carried out in a spectrometer which is suitable for MALDI-TOF spectrometry.

In the spectrometer, the ion source may be a matrix-assisted laser desorption ionisation (MALDI), an electrospray ionisation (ESI) ion source, a Fast-Atom Bombardment (FAB) ion source. Preferably, the ion source is a MALDI ion source. The MALDI ion source may be traditional MALDI source (under vacuum) or may be an atmospheric pressure MALDI (AP-MALDI) source. MALDI is a preferred ionisation method, although the use of a matrix is generally not required

In the spectrometer, the mass analyser may be a time of flight (TOF), quadrupole time of flight (Q-TOF), ion trap (IT), quadrupole ion trap (Q-IT), triple quadrupole (QQQ) Ion Trap or Time-Of-Flight Time-Of-Flight (TOFTOF) or Fourier transform ion cyclotron resonance (FTICR) mass analyser. Preferably, the mass analyser is a TOP mass analyser.

Preferably, the mass spectrometer is a MALDI-TOF mass spectrometer.

### FURTHER EMBODIMENTS

### M′ Bound to B_{P}′ by a Non-Covalent Linker

The above-mentioned embodiments of the invention may also be provided in which M′ is bound to B_{P}′ by a non-covalent bond. All the other features of the invention are the same except the groups which relate to the non-covalent bond between M′ and B_{P}′.

The non-covalent bond may be direct between M′ and B_{P}′ or may be provided by one or more binding groups present on M′ and/or B_{P}′.

Preferred non-covalent bonds are those having an association constant (Kₐ) of at least 10¹⁴ M⁻¹, preferably about 10¹⁵ M⁻¹.

In preferred embodiment, one of M′ and B_{P}′ will have a binding group comprising biotin, and the other of M′ and B_{P}′ will have a binding group comprising avidin or streptavidin.

Preferably, when the compounds of the invention comprise a non-covalent bond between M′ and B_{P}′ and a cleavable bond between C and S_{S}, Ar¹ and S_{S}, or Ar² and S_{S}, these bonds are differentially cleavable. More preferably, the non-covalent bond between M′ and B_{P}′ is not cleaved under conditions which the cleavable bond between C and S_{S}, Ar¹ and S_{S}, or Ar² and S_{S}, as appropriate, is cleaved.

L_{M} Bound to Ar¹ by More than One Bond

The above-mentioned embodiments of the invention may also be provided in which L_{M} is bound to Ar¹ by more than one covalent bond (e.g. 2 or 3 bonds) which are either single, double or triple covalent bonds, or one or more multiple bonds (e.g. double or triple covalent bonds). All the other features of the invention are the same except the groups which relate to the bond or bonds between Ar¹ and L_{M}.

Ionisation of Compounds Other than Biopolymers

In addition to biopolymers, the present invention may be used for ionising any molecule or complex of molecules which requires mass spectrum analysis. Thus, the above-mentioned embodiments of the invention may also be provided in which B_{P} is replaced by any molecule or complex having at least one group capable of reacting with M to form a covalent linkage. All the other features of the invention are the same, except group M is group capable of reacting with the molecule to be analysed.

Examples of other molecules which may be analysed in the present invention include non-biological polymers (e.g. synthetic polyesters, polyamides and polycarbonates), petrochemicals and small molecules (e.g. alkanes, alkenes, amines, alcohols, esters and amides). Amines are particularly preferred.

Examples of complexes which may be analysed in the present invention include double- and triple-stranded RNA, DNA and/or peptide nucleic acid (PNA) complexes, enzyme/substrate complexes, multimeric proteins (e.g. dimers, trimers, tetramers, pentamers, etc.), virions, etc.

Preferably, when the compound to be ionised is not a biopolymer, all embodiments of the invention (including products of formulae (I), (IIa), (IIb), (IIIa), (IIIb), (IVai), (IVaii), (IVaiii), (IVaiv), (IVbii), (IVbiii), (IVbiv), (Vai), (Vaii), (Vaiii), (Vaiv), (Vbii), (Vbiii) and (Vbiv), methods of forming an ion of formula (I) and methods of analysis) involving or relating to the compound of formula (XI) are disclaimed.

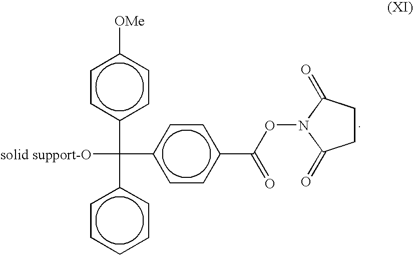

### Disclaimers

Preferably, all embodiments of the invention (including products of formulae (I) and (IIa)) involving or relating to the compound of formula (XI) are disclaimed

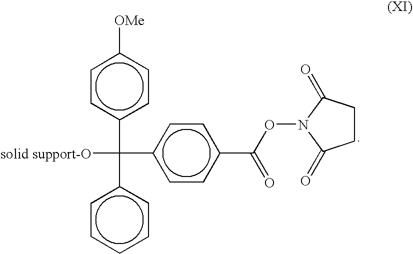

Preferably, all embodiments of the invention (including products of formulae (I) and (IIa)) involving or relating to the compound of formula (XIa) are disclaimed

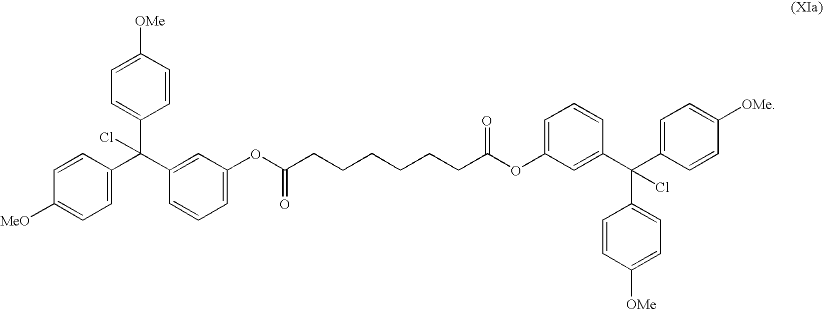

Preferably, all embodiments of the invention (including products of formulae (I) and (IIa)) involving or relating to the compound of formula (XIb) are disclaimed.

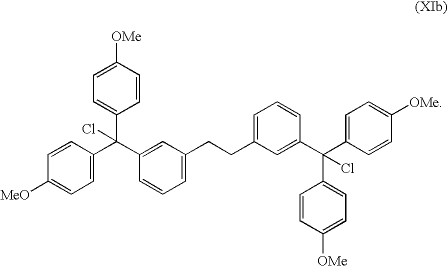

Preferably, all embodiments of the invention (including products of formulae (I) and (IIa)) involving or relating to the compound of formula (XIc) are disclaimed

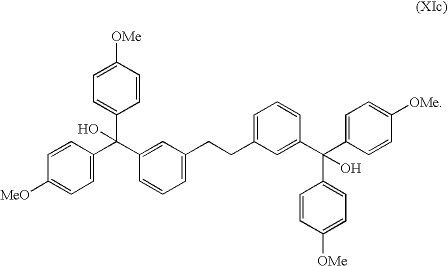

Preferably, all embodiments of the invention (including products of formulae (I) and (IIa)) involving or relating to the compound of formula (XId) are disclaimed

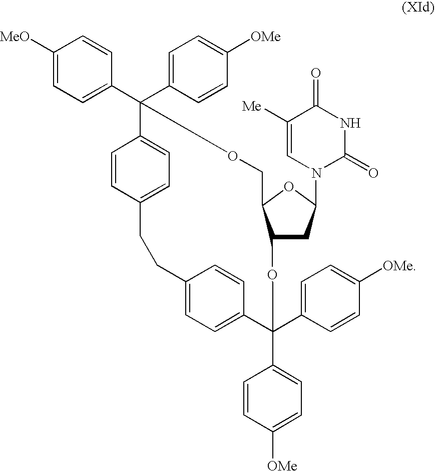

Preferably, all embodiments of the invention (including products of formulae (I) and (IIa)) involving or relating to the compound of formula (XIe) are disclaimed

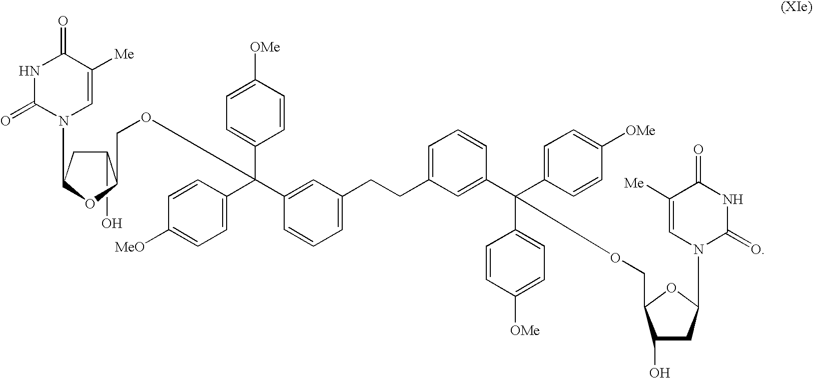

Preferably, all embodiments of the invention (including products of formulae (I) and (IIa)) involving or relating to the compound of formula (XIe) are disclaimed

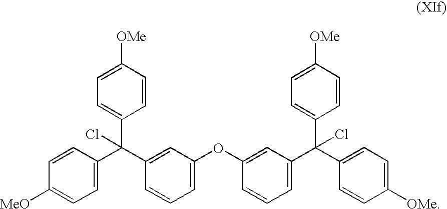

Preferably, all embodiments of the invention (including products of formulae (I) and (IIa)) involving or relating to the compound of formula (XIg-j) are disclaimed

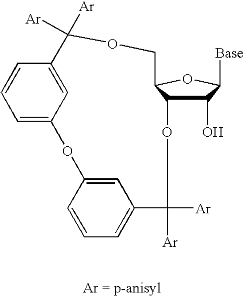

| Formula | Base |
|---|---|
| XIg | Uridine |
| XIh | N⁴-benzoyl-cytidine |
| XIi | N⁶-benzoyl-adenosine |
| XIj | N²-phenylacetyl-guanosine |

Preferably, all embodiments of the invention (including products of formulae (I) and (IIa)) involving or relating to the compound of formula (XIk-n) are disclaimed

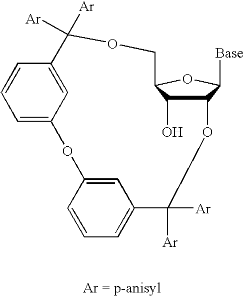

| Formula | Base |
|---|---|
| XIk | Uridine |
| XIl | N⁴-benzoyl-cytidine |
| XIm | N⁶-benzoyl-adenosine |
| XIn | N²-phenylacetyl-guanosine |

### PREFERRED EMBODIMENTS

### Definition of C

Preferably, C [symbol] bears a single positive charge such that ions of the invention are cations and the ion of formula (I) has the following structure:

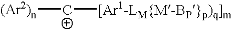

and the compounds of formulae (IIb), (IIIb), (IVbii), (IVbiii), (IVbiv), (Vbii), (Vbiii) and (Vbiv) have the structures disclosed in table 1.
n, in, p and q

For the purposes of compounds of the invention having n−1 groups Ar², n may not be less than 1.

Preferably n=2 and m=1.

Preferably p=1, 2 or 3. Preferably p=1.

Preferably q=1, 2 or 3. Preferably q=1.

Preferably n=2, m=1, p=1 and q=1. The ion of formula (I) thus has the structure:

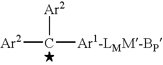

or more preferably

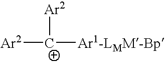

and the compounds of formulae (IIa), (IIb), (IIIa), (IIIb), (IVai), (IVaii), (IVaiii), (IVaiv), (IVbii), (IVbiii), (IVbiv), (Vai), (Vaii), (Vaiii), (Vaiv), (Vbii), (Vbiii) and (Vbiv) have the structures disclosed in table 2.

### Biopolymers

The term ‘biopolymer’ includes polymers found in biological samples, including polypeptides, polysaccharides, and polynucleotides (e.g. DNA or RNA). Polypeptides may be simple copolymers of amino acids, or they may include post-translational modifications e.g. glycosylation, lipidation, phosphorylation, etc. Polynucleotides may be single-stranded (in whole or in part), double-stranded (in whole or in part), DNA/RNA hybrids, etc. RNA may be mRNA, rRNA or tRNA.

Advantageous biopolymers are those which do not readily form a molecular ion in known MALDI-TOF MS techniques, especially those which do not form a molecular ion on illumination of laser light at 340 nm.

Biopolymers for use in the invention comprise two or more monomers, which may be the same or different as each other. Preferred biopolymers comprise at least pp monomers, where pp is 5 or more (e.g. 6, 7, 8, 9, 10, 15, 20, 25, 30, 35, 40, 45, 50, 60, 70, 80, 90, 100, 125, 150, 175, 200, 250). More preferred biopolymers comprise ppp or fewer monomers where ppp is 300 or less (e.g. 200, 100, 50).

Biopolymers may have a molecular mass of at least qq kDa, where qq=0.5 or more (e.g. 0.6, 0.7, 0.8, 0.9, 1, 1.5, 2, 3, 4, 5, 6, 7, 8, 9, 10, 15, 20, 25, 30, 40, 50, 75, 100, etc.). Preferred biopolymers are those having a molecular mass within the range of detection of a mass spectrometer. More preferred biopolymers have a molecular mass of qqq kDa or less, where qqq is 30 or less (e.g. 20, 10, 5).

Preferably, the mass, m(IX), of the fragment (IX)

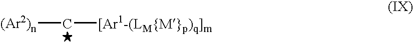

of the cation of formula (I) is significantly less than the mass, m(B_{P}′), of the biopolymer residue B_{P}′. For example the ratio m(B_{P}′)/m(IX) is preferably more than nn, where nn is at least 2 (e.g. 3, 4, 5, 10, 100, 1000, etc.).

The invention is suitable for use with purified biopolymers or mixtures of biopolymers. For example, a pure recombinant protein could be derivatised and analysed by MS, or biopolymers within a cellular lysate or extract could be derivatives and then analysed.

Preferred biopolymers are polypeptides. Particularly preferred biopolymers are polypeptides formed after proteolytic digestion of a protein.

### Biopolymers Bound to Solid Supports

In preferred embodiments of the invention the biopolymer is bound to a solid support such that it is cleavable from the solid support at least once it has been derivatised by a compound of the invention. B_{P} is thus derivatised in situ while bound to the support, and is then released. As the biopolymer is bound to the solid support, this aspect of the invention is particular relevant to methods involving compounds of formulae (IIa) and (IIb).

The biopolymer may be bound to the solid support by a covalent, ionic, hydrogen, dipole-dipole or van der Waals bond (also known as a dispersion bond or a London forces bond). The covalent, ionic, hydrogen, dipole-dipole or van der Waals bond may be direct between the biopolymer and the solid support or may be provided by one or more binding groups present on the biopolymer and/or solid support. Preferred groups are non-covalent groups.

Examples of groups which can form these types of bond, and methods for cleaving these types of bond, are set out below in connection with C—S_{S} bonds, etc.

In a particularly preferred embodiment, the solid support is provided with —(NMe₃)⁺ binding groups and the biopolymer has a net negative charge, or vice versa (i.e. the —(NMe₃)⁺ is on the biopolymer). In other preferred embodiments, the solid support is provided with anions such as carboxylate, phosphate or sulphate, or anions formed from acid groups, and the biopolymer (e.g. a histone) has a net positive charge, or vice versa.

Reactivity with Group M

The biopolymers have at least one reactive group capable of reacting with M to form a covalent linkage. Such groups typically include naturally occurring groups and groups formed synthetically on the biopolymer. I

Naturally occurring groups include lipid groups of lipoproteins (e.g. myristoyl, glycosylphosphatidylinositol, ethanolamine phosphoglycerol, palmitate, stearate, S- or N- or O-acyl groups, lipoic acid, isoprenyl, geranylgeranyl, farnesyl, etc.), amide, carbohydrate groups of N- and O-glycoproteins, amine groups (e.g. on lysine residues or at the N-terminus of a protein), hydroxyl (e.g. in β-hydroxyaspartate, β-hydroxyasparagine, 5-hydroxylysine, ¾-hydroxyproline), thiol, sulfhydryl, phosphoryl, sulfate, methyl, acetyl, formyl (e.g. on N-terminal methionines from prokaryotes), phenyl, indolyl, guanidyl, hydroxyl, phosphate, methylthio, ADP-ribosyl etc.

The reactive group is bound to the biopolymer by one or more covalent bonds (e.g. 2 or 3 bonds), which are either single, double or triple covalent bonds (preferably single bonds). Preferably, the reactive group is bound to the biopolymer by one single bond.

Groups which may be formed naturally or synthetically on the biopolymer and which are bound to the biopolymer by one bond include: —NR₂ e.g. —NHR, especially —NH₂; —SR e.g. —SH; —OR e.g. —OH; —B(R)Y; —BY₂; —C(R)₂Y; —C(R)Y₂; —CY₃; —C(=Z)Y e.g. —C(═O)Y; -Z-C(=Z)Y; —C(=Z)R e.g. —C(=Z)H, especially —C(═O)H; —C(R)(OH)OR; —C(R)(OR)₂; —S(═O)Y; -Z-S(═O)Y; —S(═O)₂Y; -Z-S(═O)₂Y; —S(═O)₃Y; -Z-S(═O)₃Y; —P(=Z)(ZR)Y e.g. —P(═O)(OH)Y; —P(=Z)Y₂; -Z-P(=Z)(ZR)Y; -Z-P(=Z)Y₂; —P(=Z)(R)Y e.g. —P(═O)(H)Y; -Z-P(=Z)(R)Y; or —N═C(=Z) e.g. —N═C(═O).

Another group which may be formed naturally or synthetically on the biopolymer and which is bound to the biopolymer by one bond is —CN.

Other groups which may be formed naturally or synthetically on the biopolymer and which are bound to the biopolymer by one bond are: —P(ZR)Y e.g. —P(OH)Y; —PY₂; -Z-P(ZR)Y; -Z-PY₂; —P(R)Y e.g. —P(H)Y; -Z-P(R)Y. A particularly preferred group is -Z-P(ZR)Y, especially a phosphoramidite group:

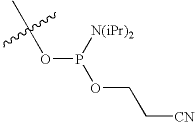

Another example of a group which may be formed naturally or synthetically on the biopolymer and which is bound to the biopolymer by one bond is —Y. In particular, when the reactive group is halo (especially iodo), the reactive group may be bound to an aliphatic or aromatic carbon.

Groups which may be formed synthetically on the biopolymer and which are bound to the biopolymer by two bonds include —N(R)— e.g. —NH—; —S—; —O—; —B(Y)—; —C(R)(Y)—; —CY₂—; —C(═O)—; —C(OH)(OR)—; —C(OR)₂—.

Groups which may be formed synthetically on the biopolymer and which are bound to the biopolymer by three bonds include

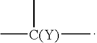

Preferred groups include nucleophilic groups, either natural or synthetic, e.g.: —NR₂ e.g. —NHR, especially —NH₂; —SR e.g. —SH; —OR e.g. —OH; —N(R)— e.g. —NH—; —S—; and —O—. The groups —NH₂, —SH and —OH are particularly preferred.

Another preferred reactive group is maleimidyl:

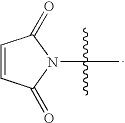

Y is independently a leaving group, including groups capable of leaving in an SN₂ substitution reaction or being eliminated in an addition-elimination reaction with the reactive group of the biopolymer B_{P}.

Preferred examples of Y include halogen (preferably iodo), C₁₋₈hydrocarbyloxy (e.g. C₁₋₈alkoxy), C₁₋₈hydrocarbyloxy substituted with one or more A, C₁₋₈heterohydrocarbyloxy, C₁₋₈heterohydrocarbyloxy substituted with one or more A, mesyl, tosyl, pentafluorophenyl, —O-succinimidyl (formula VII) or a sulfo sodium salt thereof (sulfoNHS—formula VIIa), —S-succinimidyl, or phenyloxy substituted with one or more A e.g. p-nitrophenyloxy (formula VIII) or pentafluorophenoxy (formula VIIIa).

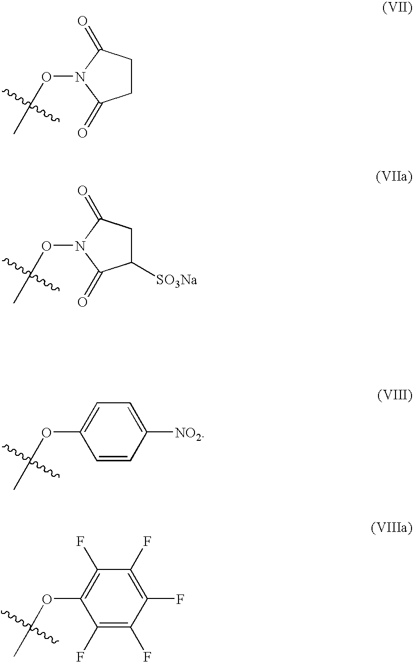

Thus, preferred reactive group on the biopolymer are:

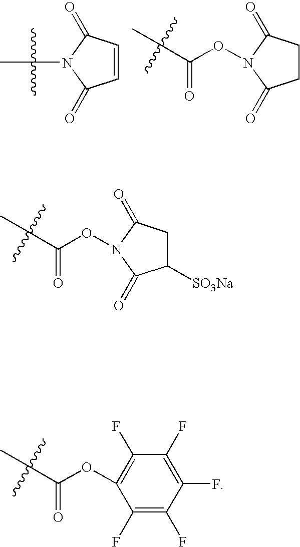

Other preferred examples of Y include -ZR. Particularly preferred examples of Y are -ZH (e.g. —OH or —NH₂) and -Z-C₁₋₈alkyl groups such as —NH—C₁₋₈alkyl groups (e.g. —NHMe) and —O—C₁₋₈alkyl groups (e.g. —O-t-butyl). Thus, preferred reactive groups are —C(O)—NH—C₁₋₈alkyl and —C(O)—O—C₁₋₈alkyl (e.g. —C(O)—O-t-butyl).

Other preferred examples of Y include -Z-ZR. Particularly preferred examples include —NR—NR₂, especially —NH—NH₂, and —ONR₂, especially —O—NH₂.

Z is independently O, S or N(R). Preferred (=Z) is (═O).

R is independently H, C₁₋₈hydrocarbyl (e.g. C₁₋₈alkyl) or C₁₋₈hydrocarbyl substituted with one or more A.

R is preferably H.

Other preferred reactive groups include —C(═O)Y, especially —C(═O)—O-succinimidyl and —C(═O)—O-(p-nitrophenyl).

In a further embodiment, the reactive group may be —Si(R)₂—Y, with Y being halo (e.g. chloro) being especially preferred. Preferred groups R in this embodiment are C₁₋₈alkyl, especially methyl. A particularly preferred reactive group in this embodiment is —Si(Me)₂Cl.

Other groups which may be formed naturally or synthetically on the biopolymer include groups capable of reacting in a cycloaddition reaction, especially a Diels-Alder reaction.

In the case of Diels-Alder reactions, the reactive group on the biopolymer is either a diene or a dienophile. Preferred diene groups are

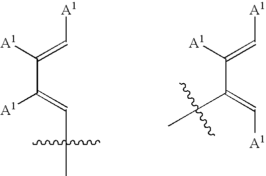

and multivalent derivatives formally formed by removal of one or more hydrogen atoms, where A¹ is —R¹ or -Z¹R¹, where R¹ and Z¹ are defined below.

Preferred dienophile groups are —CR¹═CR¹₂, —CR¹═C(R¹)A², —CA²=CR¹₂, —CA²=C(R¹)A² or —CA²=CA²₂, and multivalent derivatives formally formed by removal of one or more hydrogen atoms, where R¹ is defined below and A² is independently halogen, trihalomethyl, —NO₂, —CN, —N⁺(R¹)₂O⁻, —CO₂H, —CO₂R¹, —SO₃H, —SOR¹, —SO₂R¹, —SO₃R¹, —OC(═O)OR¹, —C(═O)H, —C(═O)R¹, —OC(═O)R¹, —OC(═O)NR¹₂, —N(R¹)C(═O)R¹, —C(═S)NR¹₂, —NR¹C(═S)R¹, —SO₂NR¹₂, —NR¹SO₂R¹, —N(R¹)C(═S)NR¹₂, or —N(R¹)SO₂NR¹₂, where R¹ is defined below. A particularly preferred dienophile group is maleimidyl.

### Group M

The group M is capable of reacting with the reactive group of the biopolymer, B_{P}, to form a covalent linkage. [Group ‘M’ is shown as ‘AFG’ in the drawings].

The group M is bound to L_{M} by one or more covalent bonds (e.g. 2 or 3 bonds, especially 2 such as

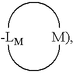

which are either single, double or triple covalent bonds (preferably single bonds). Preferably, M is bound to L_{M} by one single bond.

Alternatively, or in addition, M is bound by more than one L_{M}, such L_{M} either being attached to the same or different Ar¹ or Ar². In a preferred embodiment M is bound by more than one L_{M} from different Ar¹ or Ar², e.g.:

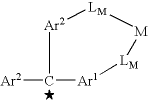

Examples of group M bound to L_{M} by one bond include —NR₂ e.g. —NHR, especially —NH₂; —SR e.g. —SH; —OR e.g. —OH; —B(R)Y; —BY₂; —C(R)₂Y; —C(R)Y₂; —CY₃; —C(=Z)Y e.g. —C(═O)Y; -Z-C(=Z)Y; —C(=Z)R e.g. —C(=Z)H, especially —C(═O)H; —C(R)(OH)OR; —C(R)(OR)₂; —S(═O)Y; -Z-S(═O)Y; —S(═O)₂Y; -Z-S(═O)₂Y; —S(═O)₃Y; -Z-S(═O)₃Y; —P(=Z)(ZR)Y e.g. —P(═O)(OH)Y; —P(=Z)Y₂; -Z-P(=Z)(ZR)Y; -Z-P(=Z)Y₂; —P(=Z)(R)Y e.g. —P(═O)(H)Y; -Z-P(=Z)(R)Y; or —N═C(=Z) e.g. —N═C(═O).

Another example of a group M bound to L_{M} by one bond is —CN.

Other examples of group M bound to L_{M} by one bond are —P(ZR)Y e.g. —P(OH)Y; —PY₂; -Z-P(ZR)Y; -Z-PY₂; —P(R)Y e.g. —P(H)Y; -Z-P(R)Y. A particularly preferred group M is -Z-P(ZR)Y, especially a phosphoramidite group:

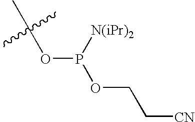

Another example of group M bound to L_{M} by one bond is —Y. In particular, when group M is halo (especially iodo), M may be bound to an aliphatic or aromatic carbon. When M is halo (e.g. iodo) and is bound to an aromatic carbon, L_{M} may, for example, be a single bond.

Examples of group M bound to L_{M} by two bonds include —N(R)— e.g. —NH—; —S—; —O—; —B(Y)—; —C(R)(Y)—; —CY₂—; —C(═O)—; —C(OH)(OR)—; —C(OR)₂—.

Examples of group M bound to L_{M} by three bonds include

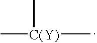

Preferred groups M include electrophilic groups, especially those susceptible to SN₂ substitution reactions, addition-elimination reactions and addition reactions, e.g. —B(R)Y; —BY₂; —C(R)₂Y; —C(R)Y₂; —CY₃; —C(=Z)Y e.g. —C(═O)Y; -Z-C(=Z)Y; —C(=Z)R e.g. —C(=Z)H, especially —C(═O)H; —C(R)(OH)OR; —C(R)(OR)₂; —S(═O)Y; -Z-S(═O)Y; —S(═O)₂Y; -Z-S(═O)₂Y; —S(═O)₃Y; -Z-S(═O)₃Y; —P(=Z)(ZR)Y e.g. —P(═O)(OH)Y; —P(=Z)Y₂; -Z-P(=Z)(ZR)Y; -Z-P(=Z)Y₂; —P(=Z)(R)Y e.g. —P(═O)(R)Y; -Z-P(=Z)(H)Y; —N═C(=Z) e.g. —N═C(═O); —B(Y)—; —C(R)(Y)—; —CY₂—; —C(═O)—; —C(OH)(OR)—; —C(OR)₂—; or

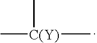

Another preferred electrophilic group M is —CN.

Still further preferred examples of group M are orthoesters, e.g. —C(OR)₃. In a preferred embodiment, the R groups are linked together to form a hydrocarbyl group, e.g. a C₁₋₈alkyl group. A preferred example of group M in this embodiment is:

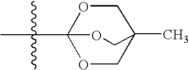

Another preferred group M is maleimido.

Y, Z and R are defined as above. Preferred Y groups when present on M are those capable of leaving in an SN₂ substitution reaction or being eliminated in an addition-elimination reaction with the reactive group of the biopolymer B_{P}.

Preferred examples of Y include halogen (preferably iodo), C₁₋₈hydrocarbyloxy (e.g. C₁₋₈alkoxy), C₁₋₈hydrocarbyloxy substituted with one or more A, C₁₋₈heterohydrocarbyloxy, C₁₋₈heterohydrocarbyloxy substituted with one or more A, mesyl, tosyl, pentafluorophenyl, —O-succinimidyl (formula VII) or a sulfo sodium salt thereof (sulfoNHS—formula VIIa), —S-succinimidyl, or phenyloxy substituted with one or more A e.g. p-nitrophenyloxy (formula VIII) or pentafluorophenoxy (formula VIIa).

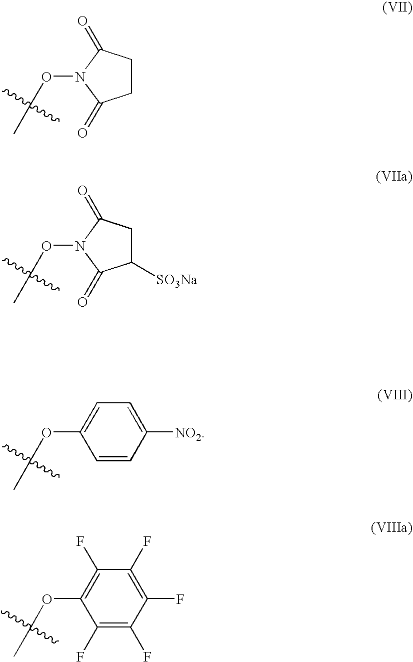

Thus, preferred groups M are:

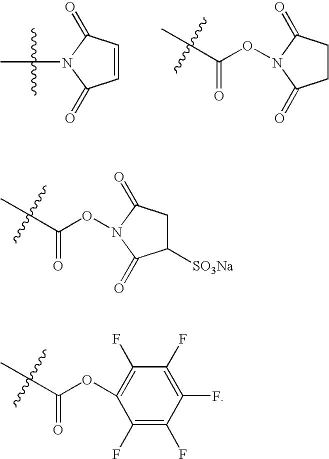

Other preferred examples of Y include -ZR. Particularly preferred examples of Y are -ZH (e.g. —OH or —NH₂) and -Z-C₁₋₈alkyl groups such as —NH—C₁₋₈alkyl groups (e.g. —NHMe) and —O—C₁₋₈alkyl groups (e.g. —O-t-butyl). Thus, preferred groups M are —C(O)—NH—C₁₋₈alkyl (e.g. —C(O)NHMe) and —C(O)—O—C₁₋₈allyl (e.g. —C(O)—O-t-butyl).

Other preferred examples of Y include -Z-ZR. Particularly preferred examples include —NR—NR₂, especially —NH—NH₂, and —ONR₂, especially —O—NH₂.

Particularly preferred groups M include —C(═O)Y, especially —C(═O)—O-succinimidyl and —C(═O)—O-(p-nitrophenyl).

In a further embodiment, M may be —Si(R)₂—Y, with Y being halo (e.g. chloro) being especially preferred. Preferred groups R in this embodiment are C₁₋₈alkyl, especially methyl. A particularly preferred group M in this embodiment is —Si(Me)₂Cl

In a further embodiment, M may be —C(Ar²)₂X. Preferred groups Ar² and X are set out below. In this embodiment it is preferred that L_{M} is a bond. A particularly preferred group M in this embodiment is:

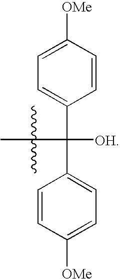

Other groups M include groups capable of reacting in a cycloaddition reaction, especially a Diels-Alder reaction.

In the case of Diels-Alder reactions, the reactive group on the biopolymer is either a diene or a dienophile. Preferred diene groups are

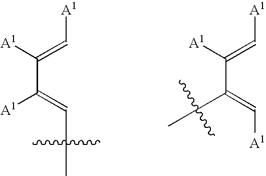

and multivalent derivatives formally formed by removal of one or more hydrogen atoms, where A¹ is —R¹ or -Z¹R¹, where R¹ and Z¹ are defined below.

Preferred dienophile groups are —CR¹═CR¹₂, —CR¹═C(R¹)A², —CA²=CR¹₂, —CA²=C(R¹)A² or —CA²=CA²₂, and multivalent derivatives formally formed by removal of one or more hydrogen atoms, where R¹ is defined below and A² is independently halogen, trihalomethyl, —NO₂, —CN, —N⁺(R¹)₂O—, —CO₂H, —CO₂R¹, —SO₃H, —SOR¹, —SO₂R¹, —SO₃R¹, —OC(═O)OR¹, —C(═O)H, —C(═O)R¹, —OC(═O)R¹, —OC(═O)NR¹₂, —N(R¹)C(═O)R¹, —C(═S)NR¹₂, —NR¹C(═S)R¹, —SO₂NR¹₂, —NR¹SO₂R¹, —N(R¹)C(═S)NR¹₂, or —N(R¹)SO₂NR¹₂, where R¹ is defined below. A particularly preferred dienophile group is maleimidyl.

Preferred examples of group M are shown in FIGS. 11A and 11B.

### Matching B_{P} and M

The reactive group on the biopolymer [shown as ‘F’ in the drawings] and the group M [shown as ‘AFG’ in the drawings] must be dependently selected in order to form the covalent linkage. For example, where the biopolymer includes the groups —NH₂, —OH or —SH, M will typically be —B(R)Y; —BY₂; —C(R)₂Y; —C(R)Y₂; —CY₃; —C(=Z)Y e.g. —C(═O)Y; -Z-C(=Z)Y; —C(=Z)R e.g. —C(=Z)H, especially —C(═O)H; —C(R)(OH)OR; —C(R)(OR)₂; —S(═O)Y; -Z-S(═O)Y; —S(═O)₂Y; -Z-S(═O)₂Y; —S(═O)₃Y; -Z-S(═O)₃Y; —P(=Z)(ZR)Y e.g. —P(═O)(OH)Y; —P(=Z)Y₂; -Z-P(=Z)(ZR)Y; -Z-P(=Z)Y₂; —P(=Z)(R)Y e.g. —P(═O)(H)Y; -Z-P(=Z)(R)Y; —N═C(=Z) e.g. —N═C(═O); —B(Y)—; —C(R)(Y)—; —CY₂—; —C(═O)—; —C(OH)(OR)—; —C(OR)₂—; or

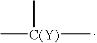

M may also be —CN.

In a preferred embodiment, one of the reactive group on the biopolymer and group M is a maleimidyl and the other will be a —SH group.

Alternatively, when the covalent linkage is to be formed by a Diels Alder reaction, one of the reactive group on the biopolymer and group M will typically be a diene and the other will be a dienophile.

Preferred covalent linkages are those produced through the reaction of the following groups:

| M | Group on B_{P} | Obtained Linkage M′-B_{P}′ |
|---|---|---|
| —C(═O)—O-succinimidyl [i.e. carboxy-NHS] | —NH₂ | —CO—NH— |
| —C(═O)—O-(p-nitrophenyl) | —NH₂ | —CO—NH— |
| —C(═O)-pentafluorophenyl | —NH₂ | —CO—NH— |
| Biotin | avidin/streptavidin | biotin-(strept)avidin |
| 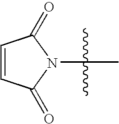 | —SH | 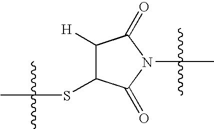 |
| —N═C═S (isothiocyanate) | —NH₂ | —NH—CS—NH— |

The covalent residue M′-B_{P}′ is the reaction product of M and B_{P}. B_{P}′ will generally be the same as B_{P} except that instead of the reactive group, B_{P}′ will have a residue of the reactive group covalently bound to the residue M′. Depending on the choice of the reactive group and the choice of M, M′ and the residue of the reactive group will typically form linkages, in the orientation L_{M}-M′-B_{P}′, including —C(R)₂Z-, -ZC(R)₂—, —C(=Z)Z-, -ZC(=Z)-, -ZC(=Z)Z-, —C(OH)(R)Z-, -ZC(OH)(R)—, —C(R)(OR)Z-, -ZC(R)(OR)—, —C(R)(OR)Z-, -ZC(R)(OR)—, —S(═O)Z-, -ZS(═O)—, -ZS(═O)Z-, —S(═O)₂Z-, -ZS(═O)₂—, -ZS(═O)₂Z-, —S(═O)₃Z-, -ZS(═O)₃—, -ZS(═O)₃Z-, —P(=Z)(ZR)Z-, -ZP(=Z)(ZR)-, -ZP(=Z)(ZR)Z-, —P(=Z)(R)Z-, -ZP(=Z)(R)—, -ZP(=Z)(R)Z—, —NH—C(=Z)-Z-, where Z and R are as defined above.

### Group M″

M″ is the same as M except that the group S_{S} is bound to a portion of M which does not form part of M′. Thus, M″ is a residue of M formable by the conjugation of M and S_{S}. However, M″ need not necessarily be formed by the conjugation of M and S_{S}.

M″---S_{S} comprises a covalent, ionic, dipole-dipole, hydrogen, or van der Waals bond. The covalent, ionic, hydrogen, dipole-dipole or van der Waals bond may be direct between M″ and S_{S} or may be provided by one or more binding groups present on M″ and/or S_{S}.

Examples of groups which can form these types of bond, and methods for cleaving these types of bond, are set out below in connection with C—S_{S} bonds, etc.

This embodiment of the invention is advantageous, since the derivativisation of the biopolymer will also release the derivatised biopolymer from the solid support. Thus, an additional step of cleaving the biopolymer from the solid support is not required.

Preferred groups M″ are groups M having a leaving group, wherein the group S_{S} is bound to the leaving group, e.g. groups M mentioned above having a leaving group Y, wherein the group S_{S} is bound to the leaving group Y.

A particularly preferred group M″ is:

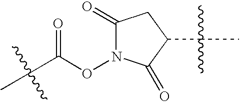

### L_{M}

Where the group L_{M} is a linker atom or group, it has a sufficient number of linking covalent bonds to link L_{M} to the group Ar¹ by a single covalent bond (or more, as appropriate) and to link L_{M} to the p instances of M (or M′, as appropriate) groups (which may be attached to L_{M} by one or more bonds).

The group L_{M} may be directly bound to the aromatic part of Ar¹, bound to one or more of the substituents A of Ar¹, or both. Preferably, L_{M} is bound directly to the aromatic part of Ar¹.

In an alternative embodiment, L_{M} may be bound to L₅.

When L_{M} is a linker atom, preferred linker atoms are O or S, particularly O.

When L_{M} is a linker group, preferred linker groups, in the orientation Ar¹-(L_{M}{M}ₚ)_{q} or Ar¹-(L_{M}{M′}ₚ)_{q}, as appropriate, are -E^{M}-, -(D^{M})ₜ-, -(E^{M}-D^{M})ₜ-, -(D^{M}-E^{M})ₜ-, -E^{M}-(D^{M}-E^{M})ₜ- or -D^{M}-(E^{M}-D^{M})ₜ-, where a sufficient number of linking covalent bonds, in addition to the covalent bonds at the chain termini shown, are provided on groups E^{M} and D^{M} for linking the p instances of M (or M′) groups.

D^{M} is independently C₁₋₈hydrocarbylene or C₁₋₈hydrocarbylene substituted with one or more A. Preferred D^{M} are C₁₋₈alkylene, C₁₋₈alkenylene and C₁₋₈alkynylene, especially C₁₋₈alkylene and C₁₋₈alkynylene, each optionally substituted with one or more A (preferably unsubstituted). A preferred substituent A is ²H. Preferred L_{M} in the orientation Ar¹-(L_{M}{M}ₚ)_{q} or Ar¹-(L_{M}{M′}ₚ)_{q}, as appropriate, are: —CH₂CH₂—; —C≡C—CH₂CH₂CH₂—; —(CH₂)₅—; —CD₂CD₂CH₂CH₂CH₂—; —C≡C—CH₂— and —CH₂CH₂CH₂—.

E^{M}, in the orientation Ar¹-(L_{M}{M}ₚ)_{q} or Ar¹-(L_{M}{M′}ₚ)_{q}, as appropriate, is independently Z^{M}-, —C(=Z^{M})-, -Z^{M}C(=Z^{M}), —C(=Z^{M})Z^{M}-, -Z^{M}C(=Z^{M})Z^{M}-, —S(═O)—, -Z^{M}S(═O)—, —S(═O)Z^{M}-, -Z^{M}S(═O)Z^{M}-, —S(═O)₂—, -Z^{M}S(═O)₂—, —S(═O)₂Z^{M}-, -Z^{M}S(═O)₂Z^{M}-, where Z^{M} is independently O, S or N(R^{M}) and where R^{M} is independently H, C₁₋₈hydrocarbyl (e.g. C₁₋₈alkyl) or C₁₋₈hydrocarbyl substituted with one or more A. Preferably E^{M} is, in the orientation Ar¹-(L_{M}{M}ₚ)_{q} or Ar¹-(L_{M}{M′}ₚ)_{q}, as appropriate, —O—, —S—, —C(═O)—, —C(═O)O—, —C(═S)—, —C(═S)O—, —OC(═S)—, —C(═O)S—, —SC(═O)—, —S(O)—, —S(O)₂—, —N(R^{M})—, —C(═O)N(R^{M})—, —C(═S)N(R^{M})—, —N(R^{M})C(═O)—, —N(R^{M})C(═S)—, —S(HO)N(R^{M})—, —N(R^{M})S(═O)—, —S(═O)₂N(R^{M})—, —N(R^{M})S(═O)₂—, —OC(═O)O—, —SC(═O)O—, —OC(═O)S—, —N(R^{M})C(═O)O—, —OC(═O)N(R^{M})—, —N(R^{M})C(═O)N(R^{M})—, —N(R^{M})C(═S)N(R^{M})—, —N(R^{M})S(═O)N(R^{M})— or —N(R^{M})S(═O)₂N(R^{M})—.

Alternative groups E^{M} to those defined above, in the orientation Ar¹-(L_{M}{M}ₚ)_{q} or Ar¹-(L_{M}{M′}ₚ)_{q}, as appropriate, are -Z^{M}-Si(R^{M})₂-Z^{M}-, —Si(R^{M})₂-Z^{M}- and -Z^{M}-Si(R^{M})₂—. The group —Si(R^{M})₂-Z^{M}- is particularly preferred. Z^{M} is preferably O. R^{M} is preferably C₁₋₈alkyl, preferably methyl. These groups E^{M} are particularly preferred in the groups -(E^{M}-D^{M})ₜ-, especially when t=1 and D^{M} is C₁₋₈alkylene. The following group is especially preferred:

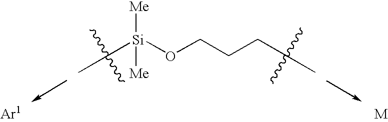

In addition to the above definition of D^{M}, D^{M} may also be C₁₋₈heterohydrocarbylene or C₁₋₈heterohydrocarbylene substituted with one or more A. In this embodiment,

C₁₋₈cycloheteroalkylene groups are particularly preferred, e.g.:

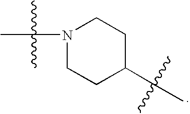

Thus, preferred L_{M} groups -D^{M}-E^{M}-D^{M}- are, in the orientation Ar¹-(L_{M}{M}ₚ)_{q} or Ar¹-(L_{M}{M′}ₚ)_{q}, as appropriate, —C₁₋₈alkylene-C(O)—C₁₋₈cycloheteroalkylene (preferably where the hetero atom is N and is bound to the carboxy), especially:

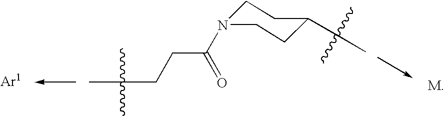

t=1 or more, e.g. from 1 to 50, 1 to 40, 1 to 30, 1 to 20 or 1 to 10. Preferably t=1, 2, 3, 4, 5, 6, 7, 8, 9, or 10.

Preferably, L_{M} links one group M (or M′) to Ar¹, M (or M′) is linked to L_{M} by a single covalent bond and therefore no additional bonds are required (e.g. L_{M}{M}₁ may be -E^{M}-{M}, -(D^{M})ₜ-{M}, -(E^{M}-D^{M})ₜ-{M}, -(D^{M}-E^{M})ₜ-{M}, -E^{M}-(D^{M}-E^{M})ₜ-{M} or -D^{M}-(E^{M}-D^{M})ₜ-{M}).

Where L_{M} includes a group which also falls within the definition of group M, the group M is preferably more reactive than the group included in L_{M}.

L_{M} is preferably -(D^{M})ₜ-, (E^{M}-D^{M})ₜ-, or -D^{M}-(E^{M}-D^{M})ₜ-.

When group L_{M} is -(D^{M})ₜ-, t is preferably 1. D^{M} is preferably C₁₋₈alkylene, preferably methylene or ethylene.

When group L_{M} is -(E^{M}-D^{M})ₜ-, or -D^{M}-(E^{M}-D^{M})ₜ-, E^{M} is preferably (in the orientation Ar¹-(L_{M}{M}ₚ)_{q} or Ar¹-(L_{M}{M′}ₚ)_{q}, as appropriate), —C(═O)N(R^{M})— (e.g. —C(═O)NH—) or O (preferably O), and D^{M} is preferably C₁₋₈alkylene, preferably ethylene, propylene, butylene or pentylene (preferably ethylene or propylene). t is preferably 1. Especially preferred L_{M} are, in the orientation Ar¹-(L_{M}{M}ₚ)_{q} or Ar¹-(L_{M}{M′}ₚ)_{q}, as appropriate, —O—CH₂CH₂CH₂— and —O—CH₂CH₂CH₂CH₂CH₂—.

Another preferred group -D^{M}-(E^{M}-D^{M})ₜ- is where D^{M} is C₁₋₈alkylene and t is 1. Preferred E^{M} in this group, in the orientation Ar¹-(L_{M}{M}ₚ)_{q} or Ar¹-(L_{M}{M′}ₚ)_{q}, as appropriate, are -Z^{M}C(=Z^{M})- (especially —N(R^{M})C(═O)—, e.g. —N(Me)C(═O)—) and —C(=Z^{M})Z^{M}- (especially —C(═O)O—). Particularly preferred L_{M} groups are:

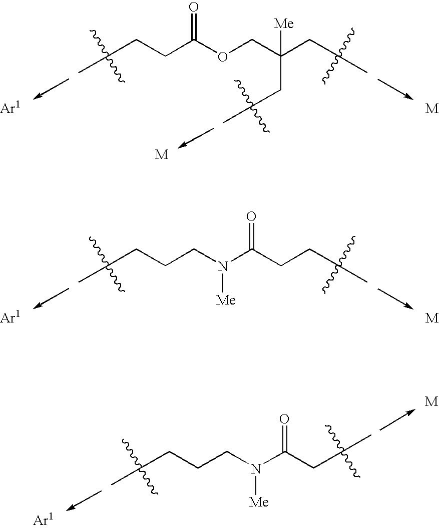

The group -(E^{M}-D^{M})ₜ- is preferred, a particularly preferred example of which is (in the orientation Ar¹-(L_{M}{M}ₚ)_{q} or Ar¹-(L_{M}{M′}ₚ)_{q}, as appropriate) —C(═O)NH—CH₂CH₂CH₂—O—CH₂CH₂—O—CH₂CH₂—O—CH₂CH₂CH₂—.

In an alternative embodiment it is preferred that L_{M} is a single covalent bond.

When Ar² is phenyl, L_{M} is preferably provided in a position ortho or para to C [symbol] . When Ar² is other than phenyl, L_{M} is preferably attached to an atom which bears the charge in at least one of the resonance structures of the ions of formula (I).

Where C [symbol] is a cation, L_{M} is preferably an electron-donating group. Where C [symbol] is an anion, L_{M} is preferably an electron-withdrawing group.

Preferred examples of L_{M} are shown in FIGS. 10A and 10B.

### C—S_{S}, S_{S}—Ar¹ and S_{S}—Ar² Bonds

C—S_{S}, S_{S}—Ar¹ and S_{S}—Ar² comprise a cleavable covalent, ionic, hydrogen, dipole-dipole or van der Waals bond (also known as a dispersion bond or a London forces bond). The covalent, ionic, hydrogen, dipole-dipole or van der Waals bond may be direct between C and S_{S}, Ar¹ and S_{S}, or Ar² and S_{S}, or may be provided by one or more binding groups present on C and/or S_{S}, Ar¹ and/or S_{S}, or Ar² and/or S_{S}, respectively.

### Covalent Bonding

Where the bond is covalent, the bond may be direct (e.g. C—S_{S}, Ar¹-S_{S} or Ar²—S_{S}, respectively) or may be provided by a linker atom or group L⁴ (e.g. C-L⁴-S_{S}, Ar¹-L⁴-S_{S} or Ar²-L⁴-S_{S}, respectively).

When L⁴ is a linker group, preferred linker groups are -E⁴-, -(D⁴)_{t″}-, -(E⁴-D⁴)_{t″}-, -(D⁴-E⁴)_{t″}-, -E⁴-(D⁴-E⁴)_{t″}- or -D⁴-(E⁴-D⁴)_{t″}-.

D⁴ is independently C₁₋₈hydrocarbylene or C₁₋₈hydrocarbylene substituted with one or more A.

E⁴ is, in the orientation C-L⁴-S_{S}, independently -Z⁴-, —C(=Z⁴)-, -Z⁴C(=Z⁴)-, —C(=Z⁴)Z⁴-, -Z⁴C(=Z⁴)Z⁴-, —S(═O)—, -Z⁴S(═O)—, —S(═O)Z⁴-, -Z⁴S(═O)Z⁴-, —S(═O)₂—, -Z⁴S(═O)₂—, —S(═O)₂Z⁴-, -Z⁴S(═O)₂Z⁴-, where Z⁴ is independently O, S or N(R⁴), and where R⁴ is independently H, C₁₋₈hydrocarbyl (e.g. C₁₋₈alkyl) or C₁₋₈hydrocarbyl substituted with one or more A. Preferably E⁴ is, in the orientation C-L⁴-S_{S}, —O—, —S—, —C(═O)—, —C(═O)O—, —C(═S)—, —C(═S)O—, —OC(═S)—, —C(═O)S—, —SC(═O)—, —S(O)—, —S(O)₂—, —N(R⁴)—, —C(═O)N(R⁴)—, —C(═S)N(R⁴)—, —N(R⁴)C(═O)—, —N(R⁴)C(═S)—, —S(═O)N(R⁴)—, —N(R⁴)S(═O)—, —S(═O)₂N(R⁴)—, —N(R⁴)S(═O)₂—, —OC(═O)O—, —SC(═O)O—, —OC(═O)S—, —N(R⁴)C(═O)O—, —OC(═O)N(R⁴)—, —N(R⁴)C(═O)N(R⁴)—, —N(R⁴)C(═S)N(R⁴)—, —N(R⁴)S(═O)N(R⁴)— or —N(R⁴)S(═O)₂N(R⁴)—.

t″=1 or more, e.g. from 1 to 50, 1 to 40, 1 to 30, 1 to 20 or 1 to 10. Preferably t″=1, 2, 3, 4, 5, 6, 7, 8, 9, or 10.

Where L⁴ includes a group which also falls within the definition of group M, the group M is preferably more reactive than the group included in L⁵.

L⁴ is preferably a linker atom, preferably O or S, particularly O.

When the solid support S_{S} is gold, L⁴ is preferably covalently attached to the S_{S} by a sulphide or disulphide group.

### Ionic Bonding

Where the bond is ionic, the bond is typically direct (e.g. C [symbol] S_{S} [symbol] , where S_{S} [symbol] is a solid support counterion to C [symbol] .

Alternatively, it may be provided by binding groups, e.g. chelating ligands, present on C or S_{S}, Ar¹ or S_{S}, or Ar² or S_{S}, respectively. In the case of C—S_{S} bonds, the chelating ligand is typically only present on S_{S} and chelates with C [symbol] .

Suitable chelating ligands which can bind anions include polyamines and cryptands.

Suitable chelating ligands which can bind cations include polyacidic compounds (e.g. EDTA) and crown ethers.

### Hydrogen Bonding

Where the bond is a hydrogen bond, the bond is usually provided by binding groups present on C or S_{S}, Ar¹ or S_{S}, or Ar² or S_{S}, respectively.

Typically, in order to form the hydrogen bond, one of C or S_{S}, Ar¹ or S_{S}, or Ar² or S_{S}, as appropriate, will have a binding group bearing one or more hydroxy, amino or thio hydrogen atoms, and the other of C or S_{S}, Ar¹ or S_{S}, or A or S_{S}, respectively, will have a binding group bearing an atom having one or more lone pair of electrons (e.g. an oxygen, sulphur or nitrogen atom). Preferably, one of C or S_{S}, Ar¹ or S_{S}, or Ar² or S_{S}, as appropriate, will have a binding group comprising biotin, and the other of C or S_{S}, Ar¹ or S_{S}, or Ar² or S_{S}, respectively, will have a binding group comprising avidin or streptavidin.

Alternatively, the hydrogen bond may be direct.

### Dipole-Dipole Bonding

Where the bond is a dipole-dipole bond, it may be formed between permanent dipoles or between a permanent dipole and an induced dipole.

Typically, in order to form the dipole-dipole bond, one of S_{S} and the compound of the invention has a permanent dipole and the other of S_{S} and the compound of the invention has an induced dipole or a permanent dipole, the attraction between the dipoles forming a dipole-dipole bond.

Preferably, S_{S} comprises binding groups (e.g. acid groups, —(NMe₃)⁺, carboxy, carboxylate, phosphate or sulphate groups) which produce a dipole at the surface of the solid support to bind the compound of the invention.

### Van der Waals Bonding

Where the bond is a van der Waals bond, the bonding is usually provided by binding groups present on C or S_{S}, Ar¹ or S_{S}, or Ar² or S_{S}, respectively.

Typically, in order to form the van der Waals bond, at least one, but preferably both, of C or S_{S}, Ar¹ or S_{S}, or Ar² or S_{S}, as appropriate, will have a hydrocarbyl or heterohydrocarbyl group (usually a large hydrocarbyl group having at least ten carbon atoms up to about 50 carbon atoms), optionally substituted with one or more A. Polyfluorinated hydrocarbyl and heterohydrocarbyl groups are particularly preferred. Typically, the hydrocarbyl or heterohydrocarbyl groups are aryl or heteroaryl groups or groups of the formula —C(R⁶)₂Ar³, —C(R⁶)(Ar³)₂ or —C(Ar³)₃, where Ar³ is independently defined the same as A² and R⁶ is H, C₁₋₈ hydrocarbyl, C₁₋₈ hydrocarbyl substituted by one or more A, C₁₋₈ heterohydrocarbyl or C₁₋₈ heterohydrocarbyl substituted by one or more A.

A preferred binding group is tetrabenzofullerene (formula X).

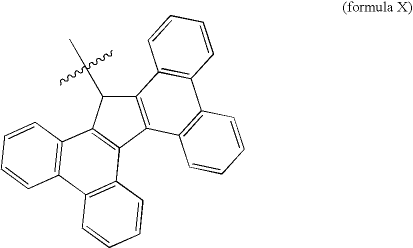

Alternatively, the van der Waals bond may be direct.

### Bond Cleavage

Preferably, the ions of formula (I) have a pKᵣ₊ value of at least zz, where zz is 0 or more (e.g. 0, 1, 2, 3, 4, 5, 6, 7, 8, 9, 10, 11, 12, 13, 14). More preferably, zz is 1 or more, still more preferably 2 or more, still more preferably 3 or more.

Preferably, the compounds of formula (IIa), (IIb), (IIIa) or (IIb) or the solid supports of formula (IVai), (IVaii), (IVaiii), (IVbii), (IVbiii), (IVaiv) or (IVbiv) provide ions of formula (I′) having a pKᵣ₊ value of at least zz, where zz is defined above.

### C—X Bonds

The C—X bonds are cleavable by irradiation, electron bombardment, electrospray, fast atom bombardment (FAB), inductively coupled plasma (ICP) or chemical ionisation. Preferably, the C—X bonds are cleavable by irradiation or chemical ionisation.

The term ‘irradiation’ includes, for example, laser illumination, in particular as used in MALDI mass spectrometry. Laser light of about 340 nm is particularly preferred because it is typically used in MALDI mass spectrometers.

The term ‘electron bombardment’ includes, for example, bombardment with electrons having energy of about 70 ev.

Chemical ionisation can be effected, for example, by treatment with acid or acidic matrices (e.g. acidic matrices used in MALDI analysis).

Preferably group X is halogen, hydroxy, C₁₋₈hydrocarbyloxy, C₁₋₈hydrocarbyloxy substituted with one or more A, C₁₋₈heterohydrocarbyloxy, C₁₋₈heterohydrocarbyloxy substituted with one or more A, mesyl, tosyl, pentafluorophenyl, —O-succinimidyl-S-succinimidyl, or phenyloxy substituted with one or more A e.g. p-nitrophenyloxy. The groups pentafluorophenyl, —O-succinimidyl, —S-succinimidyl, and p-nitrophenyloxy are particularly preferred.

Particularly preferred groups X are halogen, hydroxy, C₁₋₈hydrocarbyloxy. Especially preferred groups are hydroxy, ethoxy and chloro groups.

Other preferred groups X are alkyl ethers, e.g.:

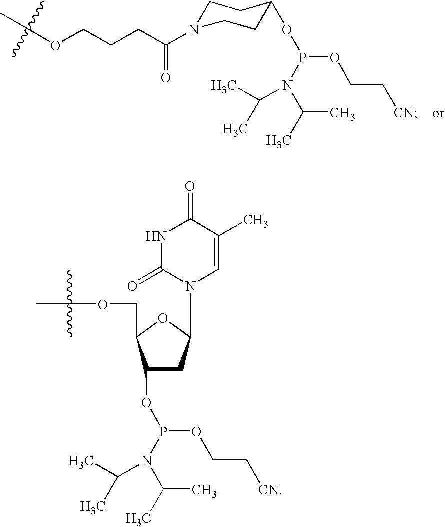

Group X may also be a -Q-oligonucleotide, where Q is O, S or N(R), where R is H, C₁₋₈hydrocarbyl or C₁₋₈hydrocarbyl substituted with one or more A. Q is preferably O.

Group X may also be a nucleoside, preferably where the nucleoside is bound via its 5′ end, e.g.:

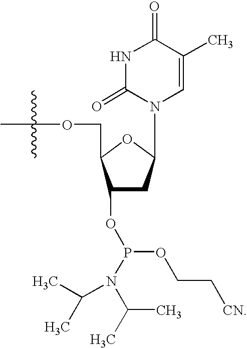

In some embodiments of the invention, where B_{P} is an antibody (particularly where it is a monoclonal antibody that recognises a tumour-associated antigen), X is not:

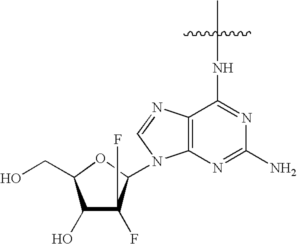

or, optionally, X is not any other 2,6-diaminopurine nucleoside prodrug group.

In some embodiments of the invention, X is not H. If X is H, preferably at least one of Ar¹ and Ar² is polycyclic, heterocyclic or unsubstituted.

Preferred examples of group X are shown in FIG. 13.

Ionic C [symbol] X [symbol] Bonds

X [symbol] is any counterion for forming salts with compounds of the invention.

X [symbol] includes ions having single charges and multiple charges. Typically ions having multiple charges will be associated with an appropriate number of compounds of formula (IIb), (IIIb), (IVbii), (IVbiii), (IVbiv), (Vbii), (Vbiii) or (Vbiv) in order balance the charge. Ions having multiple charges include doubly charged ions (e.g. SO₄²⁻) and triply charged ions. X [symbol] preferably has a single charge.

The counterion X [symbol] may be dissociated from the derivative of formula (IIb), (IIb), (IVbii), (IVbiii), (IVbiv), (Vbii), (Vbiii) or (Vbiv) by irradiation, electron bombardment, electrospray, fast atom bombardment (FAB), inductively coupled plasma (ICP) or chemical ionisation. Preferably, the counterion X [symbol] may be dissociated by irradiation.

When X [symbol] is a cation, X [symbol] is preferably H⁺.

When X [symbol] is an anion, X [symbol] is preferably, BF₆⁻ or ClO₄⁻.

It is preferred that X [symbol] is an anion.

Preferred examples of group X [symbol] are shown in FIG. 13.

### C—S_{S}, S_{S}—Ar¹ or S_{S}—Ar²

The C—S_{S}, S_{S}—Ar¹ or S_{S}—Ar² bonds are cleavable by irradiation, electron bombardment, electrospray, fast atom bombardment (FAB), inductively coupled plasma (ICP) or chemical ionisation. Preferably, the C—S_{S}, S_{S}—Ar¹ or S_{S}—Ar² bonds are cleavable by irradiation or chemical ionisation.

Where appropriate, the C—S_{S}, S_{S}—Ar¹ or S_{S}—Ar² bonds may be cleaved simultaneously or sequentially with the cleaving of the C—X bond or the dissociation of X [symbol] , as appropriate, by selection of suitable cleaving/dissociating conditions.

In one embodiment of the invention, the C—S_{S} bond in the solid support of formula (Vai) may be cleaved in sub-steps of step (iia) so that in a first sub-step a residue X (where X is the leaving group defined above) is provided and in a second subsequent sub-step the C—X bond is cleaved thereby forming the ion of formula (I). If desired, the second sub-step may be carried out substantially (e.g. seconds, minutes, hours or even days) after the first sub-step.

### Ar¹ and Ar²

### Ar²

Ar² is independently an aromatic group or an aromatic group substituted with one or more A and is preferably independently cyclopropyl, cyclopropyl substituted with one or more A, aryl, aryl substituted with one or more A, heteroaryl, or heteroaryl substituted with one or more A.

Where aryl or substituted aryl, Ar² is preferably C₆₋₃₀ aryl or substituted C₆₋₃₀ aryl. Where heteroaryl or substituted heteroaryl, Ar² is preferably C₆₋₃₀ heteroaryl or substituted C₆₋₃₀ heteroaryl.

Examples of aryl and heteroaryl are monocyclic aromatic groups (e.g. phenyl or pyridyl), fused polycyclic aromatic groups (e.g. napthyl, such as 1-napthyl or 2-napthyl) and unfused polycyclic aromatic groups (e.g. monocyclic or fused polycyclic aromatic groups linked by a single bond, a double bond, or by a —CH═CH)ᵣ— linking group, where r is one or more (e.g. 1, 2, 3, 4 or 5).

Other examples of aryl groups are monovalent derivatives of aceanthrylene, acenaphthylene, acephenanthrylene, anthracene, azulene, chrysene, coronene, fluoranthene, fluorene, as-indacene, s-indacene, indene, naphthalene, ovalene, perylene, phenalene, phenanthrene, picene, pleiadene, pyrene, pyranthrene and rubicene, which groups may be optionally substituted by one or more A.

Other examples of heteroaryl groups are monovalent derivatives of acridine, carbazole, β-carboline, chromene, cinnoline, furan, imidazole, indazole, indole, indolizine, isobenzofuran, isochromene, isoindole, isoquinoline, isothiazole, isoxazole, naphthyridine, perimidine, phenanthridine, phenanthroline, phenazine, phthalazine, purine, pyran, pyrazine, pyrazole, pyridazine, pyridine, pyrimidine, pyrrole, pyrrolizine, quinazoline, quinoline, quinolizine, quinoxaline, thiophene and xanthene, which groups may be optionally substituted by one or more A. Preferred heteroaryl groups are five- and six-membered monovalent derivatives, such as the monovalent derivatives of furan, imidazole, isothiazole, isoxazole, pyran, pyrazine, pyrazole, pyridazine, pyridine, pyrimidine, pyrrole, pyrrolizine and thiophene. The five-membered monovalent derivatives are particularly preferred, i.e. the monovalent derivatives of furan, imidazole, isothiazole, isoxazole, pyrazole, pyrrole and thiophene. The heteroaryl groups may be attached to the remainder of the compound by any carbon or hetero (e.g. nitrogen) atom.

Ar² is preferably C₆₋₃₀aryl substituted by one or more A, preferably phenyl or napthyl (e.g. 1-napthyl or 2-napthyl, especially 2-napthyl) substituted by one or more A, more preferably phenyl substituted by one or more A. When Ar² is phenyl, A is preferably provided in a position ortho or para to C [symbol] . When Ar² is other than phenyl, A is preferably attached to an atom which bears the charge in at least one of the resonance structures of the ions of formula (I).

Fused polycyclic aromatic groups, optionally substituted with one or more A, are particularly preferred.

A particularly preferred Ar² is unsubstituted pyrenyl or pyrenyl substituted with one or more A. Unsubstituted pyrenyl is preferred. The pyrenyl group may be 1-pyrenyl, 2-pyrenyl or 4-pyrenyl.

Preferred heteroaryl Ar² groups, whether substituted or unsubstituted, are pyridyl, pyrrolyl, thienyl and furyl, especially thienyl.

A preferred Ar² group is thiophenyl or thiophenyl substituted with one or more A. Unsubstituted thiophenyl is preferred. Examples of thiophenyl are thiophen-2-yl and thiophen-3-yl, with thiophen-2-yl being especially preferred.

When substituted, Ar² is preferably substituted by 1, 2 or 3 A. Ar² is preferably:

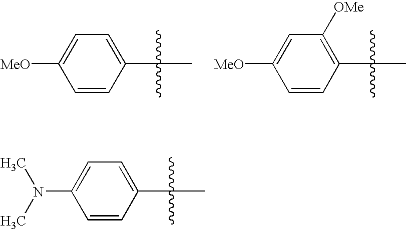

When unsubstituted, Ar² is preferably:

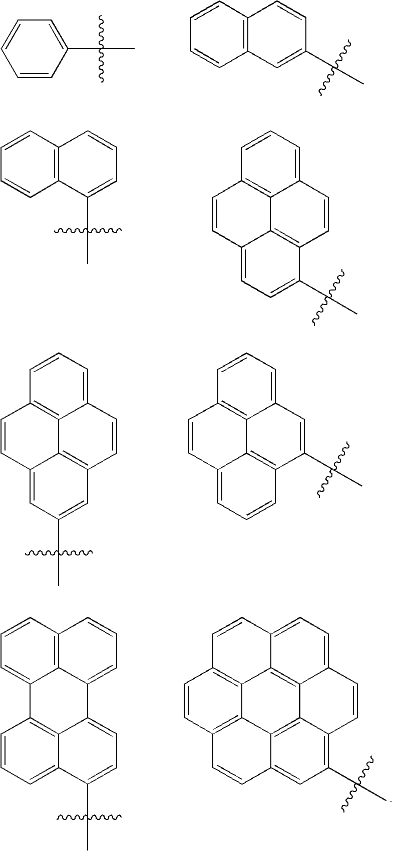

In another preferred embodiment, Ar² is cyclopropyl or cyclopropyl substituted with one or more A. Unsubstituted cyclopropyl is preferred. One or more, preferably one, of Ar² may be cyclopropyl.

Preferred examples of group Ar² are shown in FIGS. 12A and 12B.

### Ar¹

Ar¹ is independently an aromatic group or an aromatic group substituted with one or more A. The definition of Ar¹ is the same as Ar² (as defined above), except that the valency of the group Ar¹ is adapted to accommodate the q instances of the linker L_{M}. Preferred Ar² groups are also preferred Ar¹ groups, (as defined above), except that the valency of the group Ar¹ is adapted to accommodate the q instances of the linker L_{M}.

When q=1, Ar¹ is a divalent radical and is preferably independently cyclopropylene, cyclopropylene substituted with one or more A, arylene, arylene substituted with one or more A, heteroarylene, or heteroarylene substituted with one or more A.

Where arylene or substituted arylene, Ar¹ is preferably C₆₋₃₀ arylene or substituted C₆₋₃₀ arylene. Where heteroarylene or substituted heteroarylene, Ar¹ is preferably C₆₋₃₀ heteroarylene or substituted C₆₋₃₀ heteroarylene.

Examples of arylene and heteroarylene are monocyclic aromatic groups (e.g. phenylene or pyridylene), fused polycyclic aromatic groups (e.g. napthylene) and unfused polycyclic aromatic groups (e.g. monocyclic or fused polycyclic aromatic groups linked by a single bond, a double bond, or by a —(CH═CH)ᵣ— linking group, where r is one or more (e.g. 1, 2, 3, 4 or 5).

Other examples of arylene groups are polyvalent derivatives (where the valency is adapted to accommodate the q instances of the linker L_{M}) of aceanthrylene, acenaphthylene, acephenanthrylene, anthracene, azulene, chrysene, coronene, fluoranthene, fluorene, as-indacene, s-indacene, indene, naphthalene, ovalene, perylene, phenalene, phenanthrene, picene, pleiadene, pyrene, pyranthrene and rubicene, which groups may be optionally substituted by one or more A.

Other examples of heteroarylene groups are polyvalent derivatives (where the valency is adapted to accommodate the q instances of the linker L_{M}) of acridine, carbazole, β-carboline, chromene, cinnoline, furan, imidazole, indazole, indole, indolizine, isobenzofuran, isochromene, isoindole, isoquinoline, isothiazole, isoxazole, naphthyridine, perimidine, phenanthridine, phenanthroline, phenazine, phthalazine, purine, pyran, pyrazine, pyrazole, pyridazine, pyridine, pyrimidine, pyrrole, pyrrolizine, quinazoline, quinoline, quinolizine, quinoxaline, thiophene and xanthene, which groups may be optionally substituted by one or more A. Preferred heteroaryl groups are five- and six-membered polyvalent derivatives, such as the polyvalent derivatives of furan, imidazole, isothiazole, isoxazole, pyran, pyrazine, pyrazole, pyridazine, pyridine, pyrimidine, pyrrole, pyrrolizine and thiophene. The five-membered polyvalent derivatives are particularly preferred, i.e. the polyvalent derivatives of furan, imidazole, isothiazole, isoxazole, pyrazole, pyrrole and thiophene. The heteroaryl groups may be attached to the remainder of the compound by any carbon or hetero (e.g. nitrogen) atom.

Ar¹ is preferably C₆₋₃₀arylene substituted by one or more A, preferably phenylene or napthylene substituted by one or more A, more preferably phenylene substituted by one or more A. When Ar¹ is phenylene, A is preferably provided in a position ortho or para to C [symbol] . When Ar¹ is other than phenylene, A is preferably attached to an atom which bears the charge in at least one of the resonance structures of the ions of formula (I).

When substituted, Ar¹ is preferably substituted by 1, 2 or 3 A.

When unsubstituted, preferred Ar¹ are:

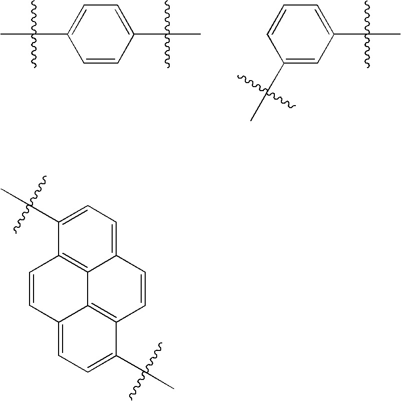

Preferred examples of group Ar¹ are shown in FIGS. 12A and 12B.

### Combinations of Ar

Optionally two or three of the groups Ar¹ and Ar² are linked together by one or more L⁵, where L⁵ is independently a single bond or a linker atom or group; and/or two or three of the groups Ar¹ and Ar² together form an aromatic group or an aromatic group substituted with one or more A.

When L⁵ is a linker group, preferred linker groups are -E⁵-, -(D⁵)ₜ-, -(E⁵-D⁵)_{t′}-, -(D⁵-E⁵)_{t′}-, -E⁵-(D⁵-E⁵)ₜ- or -D⁵-(E⁵-D⁵)_{t′}-.

D⁵ is independently C₁₋₈hydrocarbylene or C₁₋₈hydrocarbylene substituted with one or more A.

E⁵ is independently -Z⁵-, —C(=Z⁵)-, -Z⁵C(=Z⁵)-, —C(=Z⁵)Z⁵-, -Z⁵C(=Z⁵)Z⁵-, —S(═O)—, -Z⁵S(═O)—, —S(═O)Z⁵-, -Z⁵S(═O)Z⁵-, —S(═O)₂—, -Z⁵S(═O)₂—, —S(═O)₂Z⁵-, -Z⁵S(═O)₂Z⁵-, where Z⁵ is independently O, S or N(R⁵) and where R⁵ is independently H, C₁₋₈hydrocarbyl or C₁₋₈hydrocarbyl substituted with one or more A. Preferably E⁵ is —O—, —S—, —C(═O)—, —C(═O)O—, —C(═S)—, —C(═S)O—, —OC(═S)—, —C(═O)S—, —SC(═O)—, —S(O)—, —S(O)₂—, —N(R⁵)—, —C(═O)N(R⁵)—, —C(═S)N(R⁵)—, —N(R⁵)C(═O)—, —N(R⁵)C(═S)—, —S(═O)N(R⁵)—, —N(R⁵)S(═O)—, —S(═O)₂N(R⁵)—, —N(R⁵)S(═O)₂—, —OC(═O)O—, —SC(═O)O—, —OC(═O)S—, —N(R⁵)C(═O)O—, —OC(═O)N(R⁵)—, —N(R⁵)C(═O)N(R⁵)—, —N(R⁵)C(═S)N(R⁵)—, —N(R⁵)S(═O)N(R⁵)— or —N(R⁵)S(═O)₂N(R⁵)—.

t′=1 or more, e.g. from 1 to 50, 1 to 40, 1 to 30, 1 to 20 or 1 to 10. Preferably t′=1, 2, 3, 4, 5, 6, 7, 8, 9, or 10. Most preferably t′=1.

Where L⁵ includes an atom or group which also falls within the definition of group M, the group M is preferably more reactive than the group included in L⁵.

L⁵ is preferably a linker atom, preferably O or S, particularly O.

When L⁵ is a linker group, a preferred L⁵ is —N(R⁵)—.

In another embodiment in which L⁵ is a linker group, L⁵ is —S(═O)—.

When two of the groups Ar¹ and Ar² are linked together by one or more (e.g. 2, 3 or 4) L⁵, they are preferably linked together by one L⁵, preferably O.

Preferred combinations of Ar are two Ar² (e.g. two Ar² phenyl groups) linked together by one L⁵ (e.g. O or S).

Particularly preferred combinations of Ar are two Ar² phenyl groups, optionally substituted by one or more A (preferably unsubstituted), linked together by one L⁵ (e.g. O or S), where is L⁵ is ortho to C [symbol] with respect to both phenyl groups. Especially preferred combinations of two Ar² groups are:

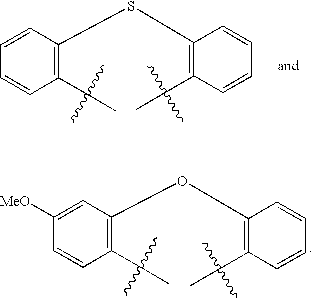

In another embodiment, at least one L_{M} is linked to an atom or group L⁵. In this embodiment, the preferred L⁵ mentioned above are, where appropriate, modified to remove substituents R⁵ in order to accommodate L_{M}, e.g. the R⁵ substituent of the group —N(R⁵)— is replaced by L_{M}. In this embodiment, the L⁵ group to which L_{M} is bound is preferably:

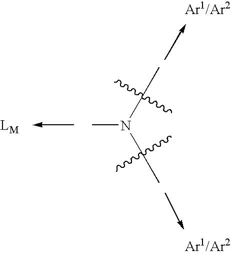

Preferred combinations of Ar¹ and/or Ar² in this embodiment are:

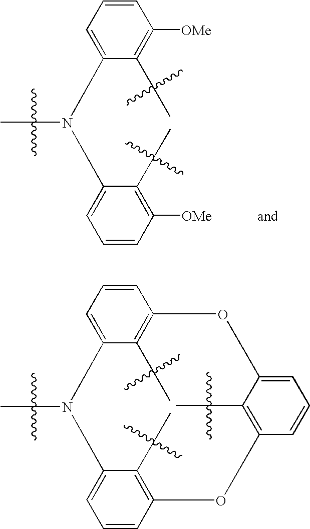

When two or three of the groups Ar¹ and Ar² together form an aromatic group or an aromatic group substituted with one or more A, the aromatic group may be a carbocyclic aromatic group or a carbocyclic aromatic group in which one or more carbon atoms are each replaced by a hetero atom. Typically, in an aromatic group in which one or more carbon atoms are each replaced by a hetero atom, up to three carbons are so replaced, preferably up to two carbon atoms, more preferably one carbon atom.

Preferred hetero atoms are O, Se, S or N⁻, more preferably O, S or N.

When two or three of the groups Ar¹ and Ar² together form an aromatic group or an aromatic group substituted with one or more A, preferred aromatic groups are C₈₋₅₀ aromatic groups.

The aromatic groups may be monocyclic aromatic groups (e.g. radicals of suitable valency derived from benzene), fused polycyclic aromatic groups (e.g. radicals of suitable valency derived from napthalene) and unfused polycyclic aromatic groups (e.g. monocyclic or fused polycyclic aromatic groups linked by a single bond, a double bond, or by a —(CH═CH)ᵣ— linking group, where r is one or more (e.g. 1, 2, 3, 4 or 5).

When two or three of the groups Ar¹ and Ar² together form a carbopolycyclic fused ring aromatic group, preferred groups are radicals of suitable valency obtained from napthalene, anthracene or phenanthracene, chrysene, aceanthrylene, acenaphthylene, acephenanthrylene, azulene, fluoranthene, fluorene, as-indacene, s-indacene, indene, phenalene, and pleiadene.

When two or three of the groups Ar¹ and Ar² together form a carbopolycyclic fused ring aromatic group in which one or more carbon atoms are each replaced by a hetero atom, preferred groups are radicals of suitable polyvalency obtained from acridine, carbazole, β-carboline, chromene, cinnoline, indole, indolizine, isobenzofuran, isochromene, isoindole, isoquinoline, naphthyridine, perimidine, phenanthridine, phenanthroline, phenazine, phthalazine, pteridine, purine, pyrrolizine, quinazoline, quinoline, quinolizine and quinoxaline.

### Substitution of Ar¹ and Ar²-Anions and Cations

When C [symbol] is a cation, A is preferably an electron-donating group, including —R¹ or -Z¹R¹, where R¹ and Z¹ are defined below. Preferably, R¹ is C₁₋₈hydrocarbyl, more preferably C₁₋₈alkyl, especially methyl. Z¹ is preferably O, S or NR¹. R¹ may be substituted with one or more S_{ub}², but is preferably unsubstituted. When C [symbol] is a cation, A is preferably —OMe, —SMe, —N(Me)₂ or Me. When C [symbol] is a cation, A, when an electron-donating group, is preferably provided (especially in relation to Ar¹ or A² being phenyl) in a position ortho or para to C [symbol] , preferably para. Furthermore, when C [symbol] is a cation, A, when an electron-withdrawing group (e.g. F), is preferably provided (especially in relation to Ar¹ or Ar² being phenyl) in a position meta to C [symbol] . Thus, preferred groups Ar¹ and Ar² are as follows:

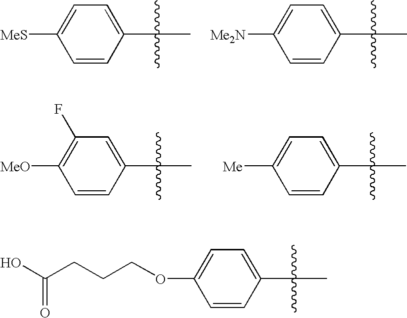

When C [symbol] is an anion, A is preferably an electron-withdrawing group, including halogen, trihalomethyl, —NO₂, —CN, —N⁺(R¹)₂O⁻, —CO₂H, —CO₂R¹, —SO₃H, —SOR¹, —SO₂R¹, —SO₃R¹, —OC(═O)OR¹, —C(═O)H, —C(═O)R¹, —OC(═O)R¹, —C(═O)NH₂, —C(═O)NR¹₂, —N(R¹)C(═O)OR¹, —N(R¹)C(═O)NR¹₂, —OC(═O)NR¹₂, —N(R¹)C(═O)R¹, —C(═S)NR¹₂, —NR¹C(═S)R¹, —SO₂NR¹₂, —NR¹SO₂R¹, —N(R¹)C(═S)NR¹₂, or —N(R¹)SO₂NR¹₂, where R¹ is defined below. When C [symbol] is an anion, A, when an electron-withdrawing group, is preferably provided (especially in relation to Ar¹ or Ar² being phenyl) in a position ortho or para to C [symbol] preferably para. Furthermore, when C [symbol] is an anion, A, when an electron-donating group, is preferably provided (especially in relation to Ar¹ or Ar² being phenyl) in a position meta to C [symbol] .

The group A may also comprise one or more isotopes of the atoms making up group A (e.g. example 60), thus, as discussed in more detail below, allowing the masses of the compounds of the invention to be varied. Preferred isotopes are ¹³C, ¹⁸O and ²H. When providing a series of compounds which differ only in their masses, ¹³C and ¹⁸O are particularly preferred as ²H atoms may cause a substantial change in the chemical properties of the compound due to the kinetic isotope effect.

### Solid Supports

‘Solid supports’ for use with the invention include polymer beads, metals, resins, columns, surfaces (including porous surfaces) and plates (e.g. mass-spectrometry plates).

The solid support is preferably one suitable for use in a mass spectrometer, such that the invention can be conveniently accommodated into existing MS apparatus. Ionisation plates from mass spectrometers are thus preferred solid supports, e.g. gold, glass-coated or plastic-coated plates. Solid gold supports are particularly preferred.

Resins or columns, such as those used in affinity chromatography and the like, are particularly useful for receiving solutions of biopolymers (purified or mixtures). For example, a cellular lysate could be passed through such a column of formula (IVai), (IVaii), (IVaiii), (IVaiv), (IVbii), (IVbiii) or (IVbiv) followed by cleavage of the support to leave compounds of formula (I).

Solid supports of formulae (IVai), (IVaii), (IVaiii), (IVaiv), (IVbii), (IVbiii) or (IVbiv) will generally present exposed groups M capable of reacting with a biopolymer, B_{P}. For MS analysis, ions preferably have a predictable mass to charge (m/e) ratio. If a biopolymer reacts with more than one M group, however, then it will carry more than one positive charge once ionised, and its m/e ratio will decrease. Advantageously, therefore, the groups M are arranged such that any biopolymer molecule will covalently link with only a single group M. Consequently, each biopolymer will, on ionisation, carry a single positive charge and thus have a predictable mass to charge ratio.

Typically, the surface density of the solid supports of (IVai), (IVaii), (IVaiii), (IVaiv), (IVbii), (IVbiii) or (IVbiv) will be provided so that a biopolymer molecule can only covalently link with one group M and thus to prevent the formation of multiply derivatised biopolymers.

### Varying the Mass of Compounds of the Invention

Within the general formulae (I), (IIa), (IIb), (IIIa), (IIIb), (IVai), (IVaii), (IVaiii), (IVaiv), (IVbii), (IVbiii), (IVbiv), (Vai), (Vaii), (Vaiii), (Vaiv), (Vbii), (Vbiii) and (Vbiv), there is much scope for variation. There is thus much scope of variation in the mass of these compounds. In some embodiments of the invention, it is preferred to use a series of two or more (e.g. 2, 3, 4, 5, 6 or more) compounds with different and defined molecular masses.

The masses of the compounds of the invention can be varied via L_{M}, Ar¹ and/or Ar². Preferably, the masses of the compounds of the invention are varied by varying A on the groups Ar¹ and/or Ar².

In this aspect of invention, compounds of the invention advantageously comprise one or more of F or I as substituents A of the groups Ar¹, Ar² or Ar³. F and I each only have one naturally occurring isotope, ¹⁹F and ¹²⁷I respectively, and thus by varying the number of F and I atoms present in the structure of the compounds, can provide a series of molecular mass labels having substantially identical shaped peaks on a mass spectrum.

Compounds of the invention may also include one or more ²H atoms, preferably as a substituent A or a part thereof of the groups L_{M}, Ar¹, Ar² or Ar³ (in particular L_{M}), in order to vary the masses of the compounds of the invention. The compounds of the invention may include isotopes of ¹³C and ¹⁸O, preferably as a substituent A or a part thereof of the groups L_{M}, Ar¹, Ar² or Ar³ (in particular Ar¹, Ar² or Ar³), in order to vary the masses of the compounds of the invention. Compounds comprising ²H, ¹³C and ¹⁸O may also be used to provide a series of molecular mass labels having substantially identical shaped peaks on a mass spectrum, by varying the number of ²H, ¹³C and ¹⁸O atoms present in the structure of the compounds. When providing a series of compounds which differ only in their masses, ¹³C and ¹⁸O are particularly preferred as ²H atoms may cause a substantial change in the chemical properties of the compound due to the kinetic isotope effect.

In order to increase the molecular mass of the compounds of the invention and to increase the number of available sites for substitution by A, especially F and I, one or more of Ar¹ and Ar² may be substituted by one or more dendrimer radicals of appropriate valency, either as substituent A or group L_{M}.

Preferred dendrimer radicals are the radicals obtained from the dendrimers of U.S. Pat. No. 6,455,071 and PAMAM dendrimers.

The compounds of the invention may advantageously be used in the method of analysing a biopolymer disclosed herein, in particular in a method for following a reaction involving a biopolymer, B_{P}, since the abundance of a species of may be determined by mass spectrometry by measuring the intensity of the relevant peak in an obtained mass spectrum.

Specifically, there is provided a method for analysing biopolymer B_{P}, comprising the steps of:
(i) reacting a first sample comprising biopolymer B_{P} with a compound of formula (IIa) or (IIb) or a solid support of formula (IVai), (IVaii), (IVaiii), (IVaiv), (IVbii), (IVbiii) or (IVbiv) at a time t₁;
(ii) reacting a second sample comprising biopolymer B_{P} with a compound of formula (IIa) or (IIb) or a solid support of formula (Vai), (IVaii), (IVaiii), (Vaiv), (IVbii), (IVbiii) or (IVbiv) at a later time t₂;
(iii) preparing and analysing cations of formula (I) from the first and second samples; and
(iv) comparing the results of the analysis from step (iii).

If levels of the biopolymer B_{P} decrease between times t₁ and t₂ then there will be a decrease in detected ion; if levels of the biopolymer B_{P} increase between times t₁ and t₂ then there will be an increase in detected ion. The effects of stimuli on transcription and/or translation can therefore be monitored.

Advantageously, different compounds of formula (IIa) or (IIb) or different solid supports of formula (IVai), (IVaii), (IVaiii), (IVaiv), (IVbii), (IVbiii) or (IVbiv) are used at different times in order to facilitate simultaneous and parallel analysis of the first and second samples. For example, if the two compounds used at times t₁ and t₂ differ only by a ¹H to ¹⁹F substitution then the relative abundance of B_{P} at the two times can be determined by comparing peaks separated by 18 units.

Advantageously, the reaction of the biopolymer with the compound of formula (IIa) or (IIb) or the solid support of formula (IVai), (IVaii), (IVaiii), (IVaiv), (IVbii), (IVbiii) or (IVbiv) will fix the biopolymer to prevent it reacting further and the steps of providing and analysing the cations may be carried out at a later convenient time. Alternatively, if the reaction of the biopolymer with the compound of formula (IIa) or (IIb) or the solid support of formula (IVai), (IVaii), (IVaiii), (IVaiv), (IVbii), (IVbiii) or (IVbiv) does not quench the reaction of the biopolymer being followed, a cation of formula (I) from the reaction product of step (i) or step (v) should be obtained as soon as possible after reaction of the biopolymer with the compound of formula (IIa) or (IIb) or the solid support of formula (IVai), (IVaii), (IVaiii), (IVaiv), (IVbii), (IVbiii) or (IVbiv).

### Compounds of Formulae (IIa) and (IIb)

The compounds of formulae (IIa) or (IIb) are available commercially or may be synthesised by known techniques.

Commercially available compounds of formulae (IIa) or (IIb) are disclosed, for example in the Molecular Probes Catalogue, 2002. Commercially available trityls, and derivatives and analogues thereof, may also be derivatised with the groups (L_{M}{M}ₚ)_{q} by known techniques.

Methods for synthesis of compounds of formula (IIa) or (IIb) useful in the present invention are described in Chem. Soc. Rev. (2003) 32, p. 3-13, scheme 2 and “1. introduction”, last two paragraphs. Groups (L_{M}{M}ₚ)_{q} are usually introduced into the intermediates and the compounds are then assembled using the appropriate pathways. Alternatively, the groups (L_{M}-{M}ₚ)_{q} may be added after assembly of the aromatic groups and α-carbon of the compounds.

Methods for synthesis of compounds of formulae (IIa) or (IIb) are also described in WO99/60007.

Further methods for synthesising the compounds of formulae (IIa) or (IIb) are described in European patent application 04 104 605.3.

Preferred compounds of formula (IIa), (IIb) and (IVai) are:

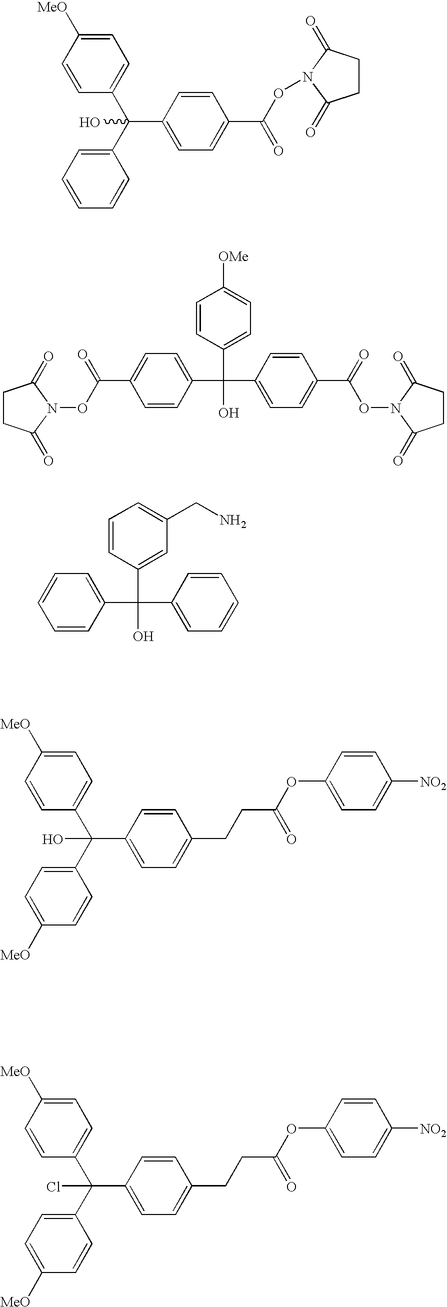

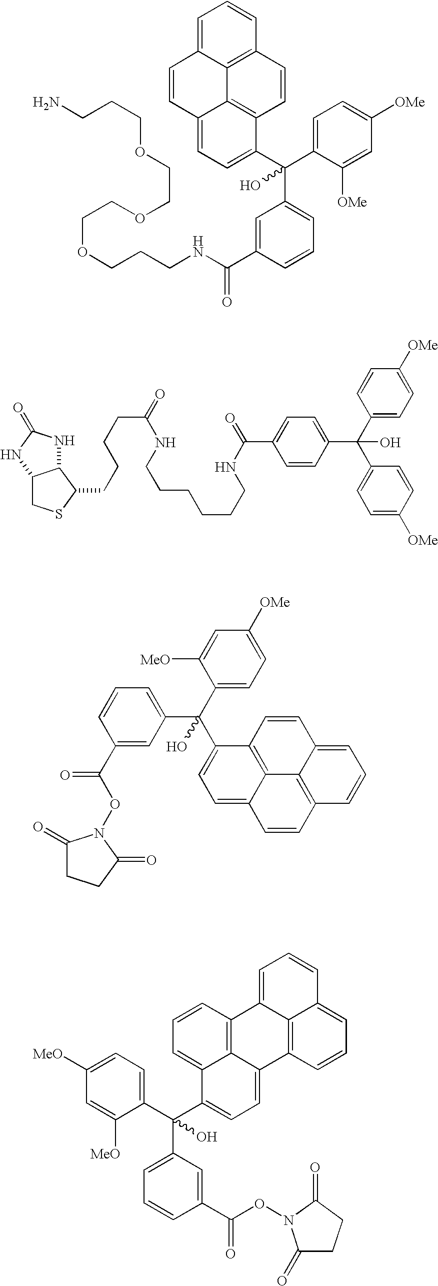

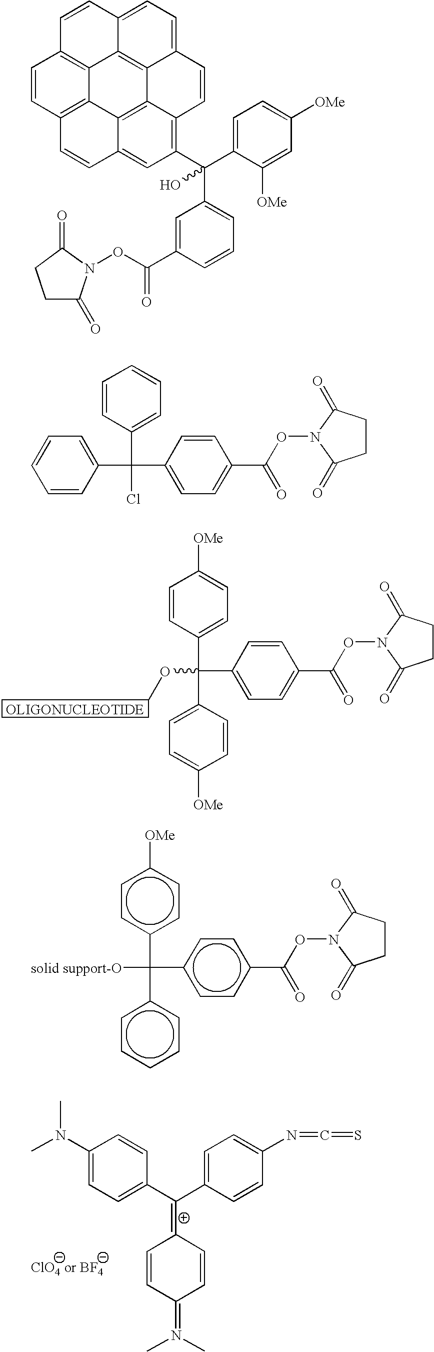

### Chemical Groups

The ions of the invention are stabilised by the resonance effect of the aromatic groups Ar¹ and Ar². The term ‘C [symbol] is a carbon atom bearing a single positive charge or a single negative charge’ therefore not only includes structures having the charge localised on the carbon atom but also resonance structures in which the charge is delocalised from the carbon atom.

The term ‘linker atom or group’ includes any divalent atom or divalent group.

The term ‘aromatic group’ includes quasi and/or pseudo-aromatic groups, e.g. cyclopropyl and cyclopropylene groups.

The term ‘halogen’ includes fluorine, chlorine, bromine and iodine.

The term ‘hydrocarbyl’ includes linear, branched or cyclic monovalent groups consisting of carbon and hydrogen. Hydrocarbyl groups thus include alkyl, alkenyl and alkynyl groups, cycloalkyl (including polycycloalkyl), cycloalkenyl and aryl groups and combinations thereof, e.g. alkylcycloalkyl, alkylpolycycloalkyl, alkylaryl, alkenylaryl, cycloalkylaryl, cycloalkenylaryl, cycloalkylalkyl, polycycloalkylalkyl, arylalkyl, arylalkenyl, arylcycloalkyl and arylcycloalkenyl groups. Preferred hydrocarbyl are C₁₋₁₄ hydrocarbyl, more preferably C₁₋₈ hydrocarbyl.

Unless indicated explicitly otherwise, where combinations of groups are referred to herein as one moiety, e.g. arylalkyl, the last mentioned group contains the atom by which the moiety is attached to the rest of the molecule.

The term ‘hydrocarbylene’ includes linear, branched or cyclic divalent groups consisting of carbon and hydrogen formally made by the removal of two hydrogen atoms from the same or different (preferably different) skeletal atoms of the group. Hydrocarbylene groups thus include alkylene, alkenylene and alkynylene groups, cycloalkylene (including polycycloalkylene), cycloalkenylene and arylene groups and combinations thereof, e.g. alkylenecycloalkylene, alkylenepolycycloalkylene, alkylenearylene, alkenylenearylene, cycloalkylenealkylene, polycycloalkylenealkylene, arylenealkylene and arylenealkenylene groups. Preferred hydrocarbylene are C₁₋₁₄ hydrocarbylene, more preferably C₁₋₈ hydrocarbylene.

The term ‘hydrocarbyloxy’ means hydrocarbyl-O—.

The terms ‘alkyl’, ‘alkylene’, ‘alkenyl’, ‘alkenylene’, ‘alkynyl’, or ‘alkynylene’ are used herein to refer to both straight, cyclic and branched chain forms. Cyclic groups include C₃₋₈ groups, preferably C₅₋₈ groups.

The term ‘alkyl’ includes monovalent saturated hydrocarbyl groups. Preferred alkyl are C₁₋₈, more preferably C₁₋₄ alkyl such as methyl, ethyl, n-propyl, i-propyl or t-butyl groups.

Preferred cycloalkyl are C₅₋₈ cycloalkyl.

The term ‘alkoxy’ means alkyl-O—.

The term ‘alkenyl’ includes monovalent hydrocarbyl groups having at least one carbon-carbon double bond and preferably no carbon-carbon triple bonds. Preferred alkenyl are C₂₋₄ alkenyl.

The term ‘alkynyl’ includes monovalent hydrocarbyl groups having at least one carbon-carbon triple bond and preferably no carbon-carbon double bonds. Preferred alkynyl are C₂₋₄ alkynyl.

The term ‘aryl’ includes monovalent aromatic groups, such as phenyl or naphthyl. In general, the aryl groups may be monocyclic or polycyclic fused ring aromatic groups. Preferred aryl are C₆-C₁₄aryl.

Other examples of aryl groups are monovalent derivatives of aceanthrylene, acenaphthylene, acephenanthrylene, anthracene, azulene, chrysene, coronene, fluoranthene, fluorene, as-indacene, s-indacene, indene, naphthalene, ovalene, perylene, phenalene, phenanthrene, picene, pleiadene, pyrene, pyranthrene and rubicene.

The term ‘alkylene’ includes divalent saturated hydrocarbylene groups. Preferred alkylene are C₁₋₄ alkylene such as methylene, ethylene, n-propylene, i-propylene or t-butylene groups.

Preferred cycloalkylene are C₅₋₈ cycloalkylene.

The term ‘alkenylene’ includes divalent hydrocarbylene groups having at least one carbon-carbon double bond and preferably no carbon-carbon triple bonds. Preferred alkenylene are C₂₋₄ alkenylene.

The term ‘alkynylene’ includes divalent hydrocarbylene groups having at least one carbon-carbon triple bond and preferably no carbon-carbon double bonds. Preferred alkynylene are C₂₋₄ alkynylene.

The term ‘arylene’ includes divalent aromatic groups, such phenylene or naphthylene. In general, the arylene groups may be monocyclic or polycyclic fused ring aromatic groups. Preferred arylene are C₆-C₁₄arylene.

Other examples of arylene groups are divalent derivatives of aceanthrylene, acenaphthylene, acephenanthrylene, anthracene, azulene, chrysene, coronene, fluoranthene, fluorene, as-indacene, s-indacene, indene, naphthalene, ovalene, perylene, phenalene, phenanthrene, picene, pleiadene, pyrene, pyranthrene and rubicene.

The term ‘heterohydrocarbyl’ includes hydrocarbyl groups in which up to three carbon atoms, preferably up to two carbon atoms, more preferably one carbon atom, are each replaced independently by O, S, Se or N, preferably O, S or N. Heterohydrocarbyl groups thus include heteroalkyl, heteroalkenyl and heteroalkynyl groups, cycloheteroalkyl (including polycycloheteroalkyl), cycloheteroalkenyl and heteroaryl groups and combinations thereof, e.g. heteroalkylcycloalkyl, alkylcycloheteroalkyl, heteroalkylpolycycloalkyl, alkylpolycycloheteroalkyl, heteroalkylaryl, alkylheteroaryl, heteroalkenylaryl, alkenylheteroaryl, cycloheteroalkylaryl, cycloalkylheteroaryl, heterocycloalkenylaryl, cycloalkenylheteroaryl, cycloalkylheteroalkyl, cycloheteroalkylalkyl, polycycloalkylheteroalkyl, polycycloheteroalkylalkyl, arylheteroalkyl, heteroarylalkyl, arylheteroalkenyl, heteroarylalkenyl, arylcycloheteroalkyl, heteroarylcycloalkyl, arylheterocycloalkenyl and heteroarylcycloalkenyl groups. The heterohydrocarbyl groups may be attached to the remainder of the compound by any carbon or hetero (e.g. nitrogen) atom.

The term ‘heterohydrocarbylene’ includes hydrocarbylene groups in which up to three carbon atoms, preferably up to two carbon atoms, more preferably one carbon atom, are each replaced independently by O, S, Se or N, preferably O, S or N. Heterohydrocarbylene groups thus include heteroalkylene, heteroalkenylene and heteroalkynylene groups, cycloheteroalkylene (including polycycloheteroalkylene), cycloheteroalkenylene and heteroarylene groups and combinations thereof, e.g. heteroalkylenecycloalkylene, alkylenecycloheteroalkylene, heteroalkylenepolycycloalkylene, alkylenepolycycloheteroalkylene, heteroalkylenearylene, alkyleneheteroarylene, heteroalkenylenearylene, alkenyleneheteroarylene, cycloalkyleneheteroalkylene, cycloheteroalkylenealkylene, polycycloalkyleneheteroalkylene, polycycloheteroalkylenealkylene, aryleneheteroalkylene, heteroarylenealkylene, aryleneheteroalkenylene, heteroarylenealkenylene groups. The heterohydrocarbylene groups may be attached to the remainder of the compound by any carbon or hetero (e.g. nitrogen) atom.

Where reference is made to a carbon atom of a hydrocarbyl or other group being replaced by an O, S, Se or N atom, what is intended is that:

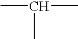

is replaced by

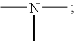

—CH═ is replaced by —N═; or
—CH₂— is replaced by —O—, —S— or —Se—.

The term ‘heteroalkyl’ includes alkyl groups in which up to three carbon atoms, preferably up to two carbon atoms, more preferably one carbon atom, are each replaced independently by O, S, Se or N, preferably O, S or N.

The term ‘heteroalkenyl’ includes alkenyl groups in which up to three carbon atoms, preferably up to two carbon atoms, more preferably one carbon atom, are each replaced independently by O, S, Se or N, preferably O, S or N.

The term ‘heteroalkynyl’ includes alkynyl groups in which up to three carbon atoms, preferably up to two carbon atoms, more preferably one carbon atom, are each replaced independently by O, S, Se or N, preferably O, S or N.

The term ‘heteroaryl’ includes aryl groups in which up to three carbon atoms, preferably up to two carbon atoms, more preferably one carbon atom, are each replaced independently by O, S, Se or N, preferably O, S or N. Preferred heteroaryl are C₅₋₁₄heteroaryl. Examples of heteroaryl are pyridyl, pyrrolyl, thienyl or furyl.

Other examples of heteroaryl groups are monovalent derivatives of acridine, carbazole, β-carboline, chromene, cinnoline, furan, imidazole, indazole, indole, indolizine, isobenzofuran, isochromene, isoindole, isoquinoline, isothiazole, isoxazole, naphthyridine, perimidine, phenanthridine, phenanthroline, phenazine, phthalazine, purine, pyran, pyrazine, pyrazole, pyridazine, pyridine, pyrimidine, pyrrole, pyrrolizine, quinazoline, quinoline, quinolizine, quinoxaline, thiophene and xanthene. Preferred heteroaryl groups are five- and six-membered monovalent derivatives, such as the monovalent derivatives of furan, imidazole, isothiazole, isoxazole, pyran, pyrazine, pyrazole, pyridazine, pyridine, pyrimidine, pyrrole, pyrrolizine and thiophene. The five-membered monovalent derivatives are particularly preferred, i.e. the monovalent derivatives of furan, imidazole, isothiazole, isoxazole, pyrazole, pyrrole and thiophene.

The term ‘heteroalkylene’ includes alkylene groups in which up to three carbon atoms, preferably up to two carbon atoms, more preferably one carbon atom, are each replaced independently by O, S, Se or N, preferably O, S or N.

The term ‘heteroalkenylene’ includes alkenylene groups in which up to three carbon atoms, preferably up to two carbon atoms, more preferably one carbon atom, are each replaced independently by O, S, Se or N, preferably O, S or N.

The term ‘heteroalkynylene’ include alkynylene groups in which up to three carbon atoms, preferably up to two carbon atoms, more preferably one carbon atom, are each replaced independently by O, S, Se or N, preferably O, S or N.

The term ‘heteroarylene’ includes arylene groups in which up to three carbon atoms, preferably up to two carbon atoms, more preferably one carbon atom, are each replaced independently by O, S, Se or N, preferably O, S or N. Preferred heteroarylene are C₅₋₁₄heteroarylene. Examples of heteroarylene are pyridylene, pyrrolylene, thienylene or furylene.

Other examples of heteroarylene groups are divalent derivatives (where the valency is adapted to accommodate the q instances of the linker L_{M}) of acridine, carbazole, β-carboline, chromene, cinnoline, furan, imidazole, indazole, indole, indolizine, isobenzofuran, isochromene, isoindole, isoquinoline, isothiazole, isoxazole, naphthyridine, perimidine, phenanthridine, phenanthroline, phenazine, phthalazine, purine, pyran, pyrazine, pyrazole, pyridazine, pyridine, pyrimidine, pyrrole, pyrrolizine, quinazoline, quinoline, quinolizine, quinoxaline, thiophene and xanthene. Preferred heteroarylene groups are five- and six-membered divalent derivatives, such as the divalent derivatives of furan, imidazole, isothiazole, isoxazole, pyran, pyrazine, pyrazole, pyridazine, pyridine, pyrimidine, pyrrole, pyrrolizine and thiophene. The five-membered divalent derivatives are particularly preferred, i.e. the divalent derivatives of furan, imidazole, isothiazole, isoxazole, pyrazole, pyrrole and thiophene.

### Substitution

A is independently a substituent, preferably a substituent S_{ub}¹. Alternatively, A may be ²H.

S_{ub}¹ is independently halogen, trihalomethyl, —NO₂, —CN, —N⁺—(R¹)₂O⁻, —CO₂H, —CO₂R¹, —SO₃H, —SOR¹, —SO₂R¹, —SO₃R¹, —OC(═O)OR¹, —C(═O)H, —C(═O)R¹, —OC(═O)R¹, —NR¹₂, —C(═O)NH₂, —C(═O)NR¹₂, —N(R¹)C(═O)OR¹, —N(R¹)C(═O)NR¹₂, —OC(═O)NR¹₂, —N(R¹)C(═O)R¹, —C(═S)NR¹₂, —NR¹C(═S)R¹, —SO₂NR¹₂, —NR¹SO₂R¹, —N(R¹)C(═S)NR¹₂, —N(R¹)SO₂NR¹₂, —R¹ or -Z¹R¹.

Z¹ is O, S, Se or NR¹.

R¹ is independently H, C₁₋₈hydrocarbyl, C₁₋₈hydrocarbyl substituted with one or more S_{ub}², C₁₋₈ heterohydrocarbyl or C₁₋₈heterohydrocarbyl substituted with one or more S_{ub}²

S_{ub}² is independently halogen, trihalomethyl, —NO₂, —CN, —N⁺(C₁₋₆alkyl)₂O; —CO₂H, —CO₂C₁₋₆alkyl, —SO₃H, —SOC₁₋₆alkyl, —SO₂C₁₋₆alkyl, —SO₃C₁₋₆alkyl, —OC(═O)OC₁₋₆alkyl, —C(═O)H, —C(═O)C₁₋₆alkyl, —OC(═O)C₁₋₆alkyl, —N(C₁₋₆alkyl)₂, —C(═O)NH₂, —C(═O)N(C₁₋₆alkyl)₂, —N(C₁₋₆alkyl)C(═O)O(C₁₋₆alkyl), —N(C₁₋₆alkyl)C(═O)N(C₁₋₆alkyl)₂, —OC(═O)N(C₁₋₆alkyl)₂, —N(C₁₋₆alkyl)C(═O)C₁₋₆alkyl, —C(═S)N(C₁₋₆alkylN, —N(C₁₋₆alkyl)C(═S)C₁₋₆alkyl, —SO₂N(C₁₋₆alkyl)₂, —N(C₁₋₆alkyl)SO₂C₁₋₆alkyl, —N(C₁₋₆alkyl)C(═S)N(C₁₋₆alkyl)₂, —N(C₁₋₆alkyl)SO₂N(C₁₋₆alkyl)₂, C₁₋₆alkyl or -Z¹C₁₋₆alkyl.

Where reference is made to a substituted group, the substituents are preferably from 1 to 5 in number, most preferably 1.

However, molecular mass labels of the invention will generally comprise 1 or more, typically between 1 and 100 (e.g. 1 to 50, preferably 1 to 20) substituents S_{ub}¹ or S_{ub}², typically F or I, in order to vary the masses of the molecular mass labels.

Preferred examples of substituent A are shown in FIG. 14.

### Miscellaneous

A may optionally be a monovalent dendrimer radical or a monovalent dendrimer radical substituted with one or more substituents S_{ub}¹.

### General

The term “comprising” means “including” as well as “consisting” e.g. a composition “comprising” X may consist exclusively of X or may include something additional e.g. X+Y.

The term “about” in relation to a numerical value x means, for example, x±10%.

The word “substantially” does not exclude “completely” e.g. a composition which is “substantially free” from Y may be completely free from Y. Where necessary, the word “substantially” may be omitted from the definition of the invention.

### Tables

TABLE 1
C [symbol] is a cation

| Formula | Structure |
|---|---|
| Formula (I) | 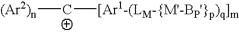 |
| Formula (IIb) | 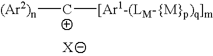 |
| Formula (IIIb) | 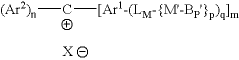 |
| Formula (IVbii) | 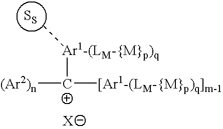 |
| Formula (IVbiii) | 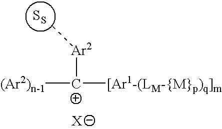 |
| Formula (IVbiv) | 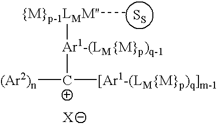 |
| Formula (Vbii) | 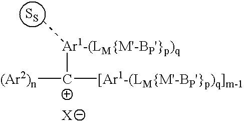 |
| Formula (Vbiii) | 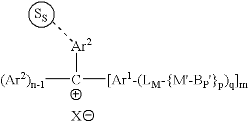 |
| Formula (Vbiv) | 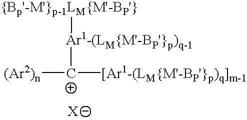 |

TABLE 2
n = 2, m = 1, p = 1 and q = 1

| Formula | Structure |
|---|---|
| Formula (I) | 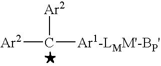 |
| Formula (IIa) | 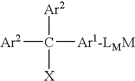 |
| Formula (IIb) | 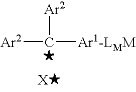 |
| Formula (IIIa) | 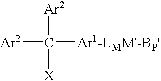 |
| Formula (IIIb) | 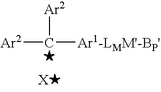 |
| Formula (IVai) | 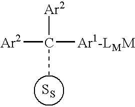 |
| Formula (IVaii) | 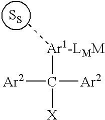 |
| Formula (IVaiii) | 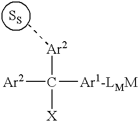 |
| Formula (IVaiv) | 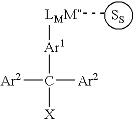 |
| Formula (IVbii) | 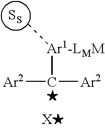 |
| Formula (IVbiii) | 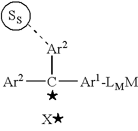 |
| Formula (IVbiv) | 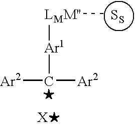 |
| Formula (Vai) | 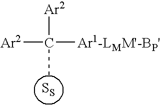 |
| Formula (Vaii) | 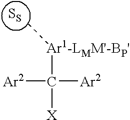 |
| Formula (Vaiii) | 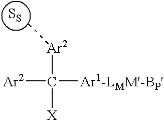 |
| Formula (Vaiv) | 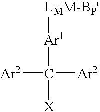 |
| Formula (Vbii) | 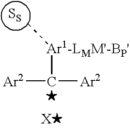 |
| Formula (Vbiii) | 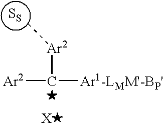 |
| Formula (Vbiv) | 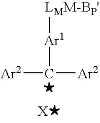 |

### BRIEF DESCRIPTION OF THE DRAWINGS

FIG. 1 demonstrates conceptually the effect of the signal on a mass spectrum by a compound of formula (IIa) or (IIb) of the invention. Free biopolymer, such as a peptide, has poorer desorption properties characterised by a smaller peak on the left of mass-spectrum whereas desorption improves when the same molecule is conjugated to a compound of the invention.

FIG. 2 shows the steps of biopolymer with a compound of formula (IVai). The derivativisation of a biopolymer with a compound of the invention can be carried out more conveniently by utilising the solid phase-based format, whereby the compound is temporarily covalently attached to a solid support. This eliminates all the separation steps associated with homogenous approach as the only additional step required would be a washing step. The solid support can be a resin, a surface or a porous surface. Alternatively, the solid support may be a mass-spectrometry sample plate, which dramatically decreases the sample preparation time. Both gold, glass- and plastic-coated plates are compatible with this approach.

FIG. 3 shows the steps of ‘reverse’ biopolymer derivativisation on a covalent solid support whereby the release of the biopolymer derivative happens simultaneously with the derivativisation process. The process is applicable M groups involving leaving groups.

FIG. 4 shows the steps of biopolymer derivativisation on an ionic solid support.

FIG. 5 shows of the steps of solid support-assisted biopolymer derivativisation. The biopolymer is first trapped onto a solid support and then labelled with a compound of formula (IIa) or (IIb). An advantage of this technique is that a preliminary sample enrichment occurs, since not all of the biopolymer in the sample will stick to the solid support surface.

FIG. 6 shows the mass spectrum obtained when analysing an Gly-Gly-O-acyl dipeptide conjugated with a trityl compound of the invention.

FIG. 7 shows the mass spectrum obtained when analysing a conjugate of a peptide with a trityl compound of the invention.

FIG. 8 compared the mass spectra of a BSA digest without (8A) and with (8B) labelling.

FIG. 9 shows the mass spectrum obtained when analysing a mixture of trityl-labelled amines.

FIGS. 10A and 10B show preferred examples of group L_{M}.

FIGS. 11A and 11B show preferred examples of group M.

FIGS. 12A and 12B shows preferred examples of groups Ar¹ and Ar².

FIG. 13 shows preferred examples of groups X and X [symbol] .

FIG. 14 shows preferred examples of substituent group A.

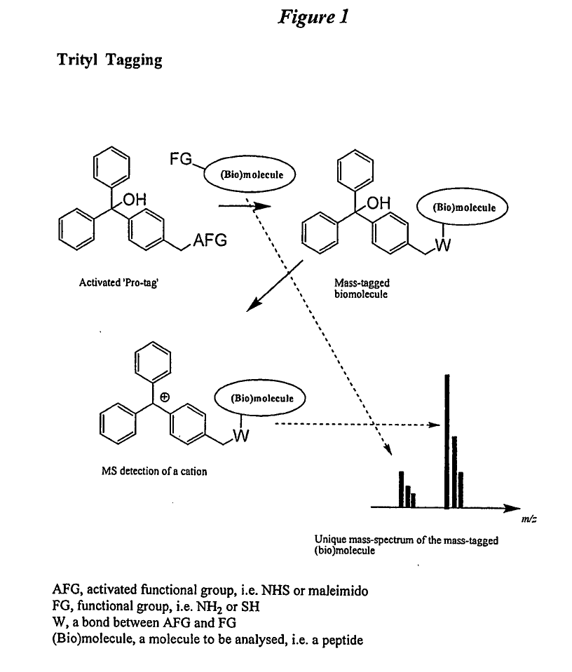

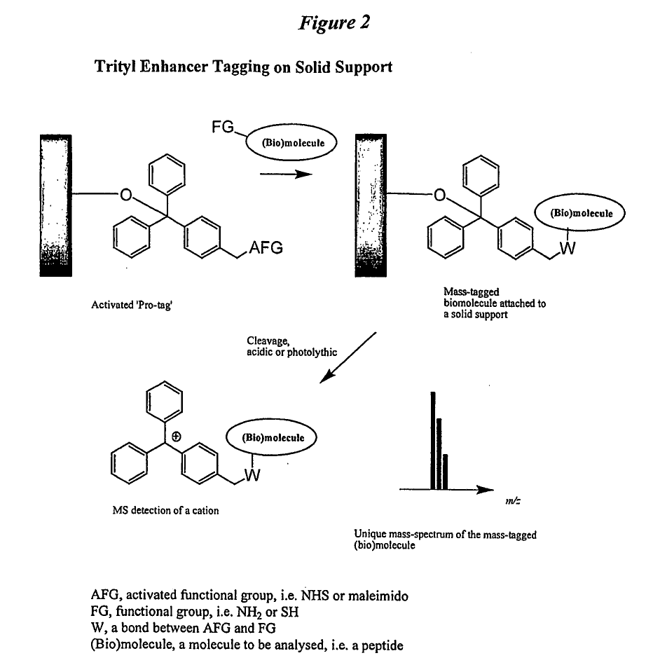

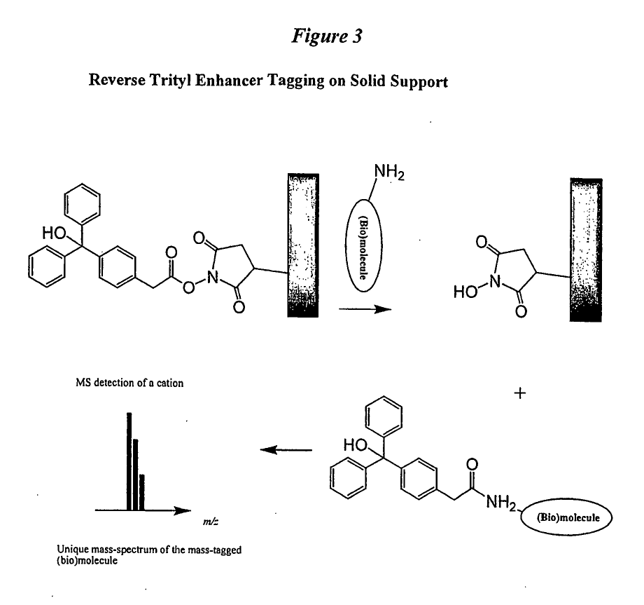

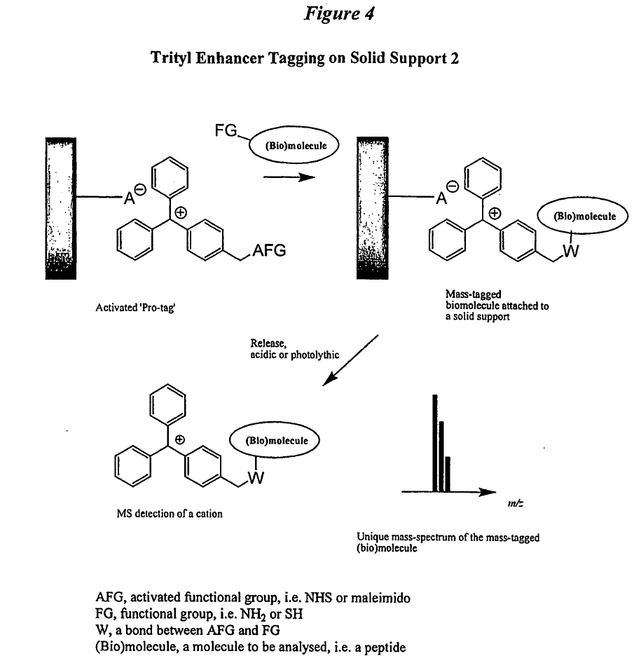

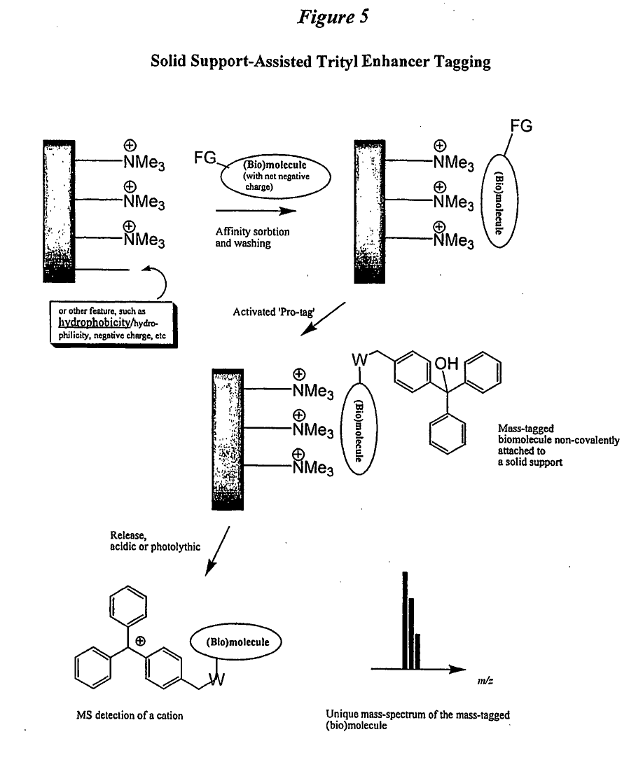

### MODES FOR CARRYING OUT THE INVENTION

### Materials and Methods

The solid supports were Tenta Gel Macrobeads OH and NH₂, 280-320 microns, Rapp Polymer. (MA)LDI-TOF mass-spectra were recorded on a PE-ABI Voyager™ Elite Reflectron Delayed Extraction Instrument. TLC were carried out with Merck silica gel (Kieselgel 60 F₂₅₄ precoated plates and Kieselgel 60 0.040-0.063 mm). HPLC was carried out on a Waters system (Milford, Mass., USA). Phosphoroamidite couplings were carried out in an ABI 394 DNA/RNA synthesiser. Chemicals and solvents were from Sigma/Aldrich/Fluka (USA), and BDH/Merck.

### Example 1

### Conjugation of a Trityl Tag (in Solution Phase) with Solid Support-Bound Biopolymer

A 15mer poly-T oligonucleotide was synthesised on an ABI 394 DNA synthesiser using a T CPG support according to standard protocols of phosphoramidite chemistry on 0.2 μmol scale. After the last coupling, a MMTr-protected ‘aminolink’ phosphoramidite (Glen Res., USA) was added to a growing chain and deprotected using standard deblocker (2% DCA in DCM). The column was removed from the synthesiser, and after 10 min wash with acetonitrile it was attached to two 5 ml syringes and washed with a 0.1M solution of NHS-activated 4,4′-dimethoxy-4″-carboxyethyl trityl for 10 min at RT. The column was then washed with (3×10 ml) acetonitrile, placed on a DNA synthesiser and deprotected with ammonia according to standard protocols. The residue obtained after the evaporation was dissolved in 0.1 ml of 2M LiClO₄ and precipitated from cold acetone (1.5 ml). The precipitate was washed with 0.5 ml of acetone and dried.

### Example 2

### Homogenous Conjugation of a Trityl with Non-Polymeric Ligands

A solution of NHS-activated 4,4′-dimethoxy-4″-carboxyethyl trityl (0.1M) in THF/dioxane (1:1) was mixed with a solution (0.5-1M) of an amine or of a mixture of amines (for example, propyl amine, butyl amine, pentyl amine, hexyl amine and phenethyl amine), typically 10 ml of a solution of an activated trityl with 5 ml of an amine solution. The mixtures were purified on prep-TLC (2 mm-thick glass plates with UV254 indicator, Analtech/Aldrich-Sigma), typically in chloroform with 0.5% triethylamine. The areas containing the desired products were scratched off the plate, and the conjugates or the mixtures thereof were eluted using same solvent with 2-5% MeOH, filtered through a layer of glass wool, evaporated and dried.

### Example 3

### Homogenous Conjugation of a Nhs-Activated Trityl with Polymeric Ligands

A peptide, an oligonucleotide, or any other biopolymer containing a (primary) amino group, is dissolved in a mixture of water and acetonitrile depending on its solubility, typically 20-50% of water in CH₃CN. Non-aminogroup-containing buffers (ie. 50 mM sodium phosphate, 0.15 M NaCl, pH 7.2, or a bicarbonate buffer, but an additional desalting step may then need to be introduced to cut off the metal ions prior to mass-spectrometry) can be used to keep the pH at between 7-9. For particularly poorly soluble ligands other solvents may be used such as THF, DMSO, etc.

A solution of NHS-activated 4,4′-dimethoxy-4″-carboxyethyl trityl in acetonitrile or THF is added in approx. 5-10 times excess compared to an amine component. Conjugation usually reaches the maximum yield over 2-4 hours of reaction time. The conjugate formed can be analysed by MS directly, or after HPLC-purification.

### Example 4

### Conjugation of a Solid Phase-Immobilised Nhs-Activated Trityl Tag with a Ligand

A Solid Phase-Immobilised NHS-Activated Trityl Tag was prepared by either method 1 or method 2.

Method 1: A NHS-Activated 4,4′-dimethoxy-4″-carboxyethyl trityl tag was covalently attached to hydroxyl groups of 200 μm Rapp Polymer beads by shaking the suspension of 100 mg of the resin in 5 ml of 0.1 M solution of trityl chloride tag in dry pyridine at +4° C. for 3 hours and then washing the resin with pyridine and acetonitrile and drying in vacuo.

Method 2. A 5′-tritylated thymidine phosphoramidite was prepared from NHS-activated 4,4′-dimethoxy-4″-carboxyethyl trityl chloride in a standard way [M. J. Gait, Oligonucleotide Synthesis: A Practical Approach, IRL, Oxford, 1984]. The Rapp Polymer beads (2×40 mg) were placed in two 1 micromol scale DNA synthesis columns (Glen Res.). The first column was coupled with the said phosphoramidite on an ABI DNA synthesiser using manual supply of reagents (0.1M solution of a phosphoramidite and other standard phosphoramidite synthesis reagents) with a coupling step of 15 min. The second column was first derivatised with a trebler phosphoramidite (Glen Res.) according to the manufacturer's protocols and then coupled with the trityl tag-containing phosphoramidite as described for the first column. Both columns were excessively washed with acetonitrile.

The trityl loading of the solid supports produced by either method was determined spectrophotometrically (absorbance measurements at 490 nm) to be 0.21 mmol/g for a straight attachment and 0.39 mmol/g for a tritylation on top of the trebling synthon. (The hydroxyl group loading of the Rapp polymer used was 0.25 mmol/g).

To the solid support prepared as described above, a mixture of compounds to be labelled (typically peptides) is added, typically in a mixture of 20-50% water in acetonitrile. After incubation, with occasional shaking, for 60-120 min the resin is washed with several volumes of the same solvent, and the conjugated products are cleaved off the resin, typically by adding 0.5-2% TFA in appropriate solvent. The collected sample is then analysed by MS.

### Example 5

### Mass Spectrometry Analysis of a Derivatised Gly-Gly Dipeptide

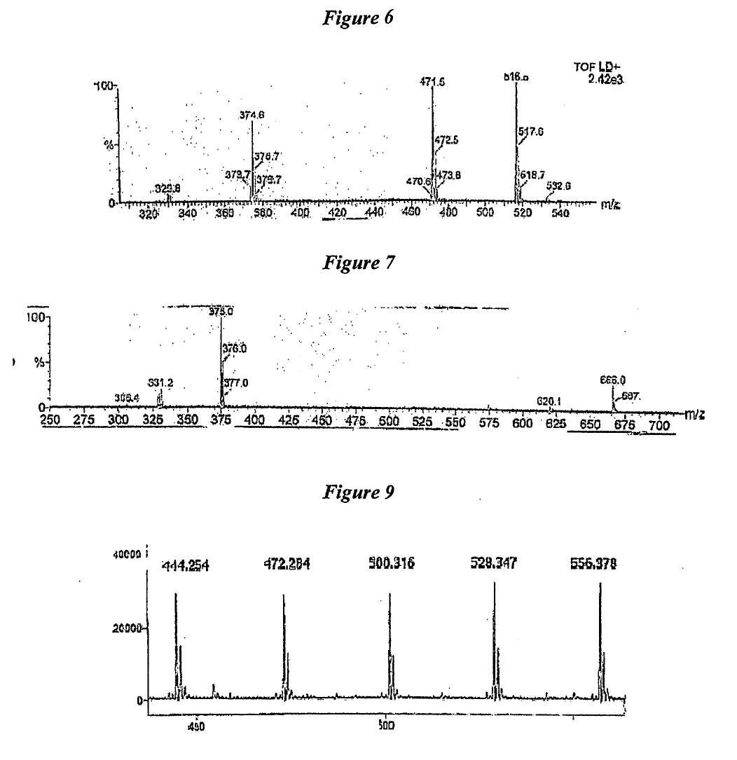

FIG. 6 shows the mass spectrum obtained from a compound of the invention comprising a derivatised Gly-Gly-O-acyl dipeptide biopolymer.

The ion of formula (I) containing the derivatised Gly-Gly-O-acyl biopolymer is observed at the peak at molecular weight 516.5. There was no peak corresponding to the free dipeptide.

The fragment of formula (VI), in which the derivatised Gly-Gly-O-acyl biopolymer has been lost, is observed at the peak at the molecular weight 374.6.

### Example 6

### Mass Spectrometry Analysis of a Derivatised Peptide

FIG. 7 shows the mass spectrum obtained from a compound of the invention comprising a derivatised peptide biopolymer. The free peptide had a molecular weight of 310.

The ion of formula (I) containing the derivatised peptide biopolymer is observed at the peak at molecular weight 665.0.

The fragment of formula (VI), in which the derivatised peptide has been lost, is observed at the peak at the molecular weight 375.0.

Significantly, there is only a very small peak at molecular weight 310, where a peak corresponding to the free biopolymer would be found. The relative size of the peaks at 665.0 and 310 thus demonstrate the significantly improved ionisability of the compounds of the invention compared with free biopolymer.

### Example 7

### Spectral Improvement by Trityls

Three proteins (BSA, β-casein and ADH) were digested with trypsin and the resulting peptides analysed by MALDI-TOF mass spectrometry with or without derivatisation. The number of peptides identified for each protein is shown below. The theoretical total number of peptides that would be produced by trypsin digestion of each protein was calculated in silico and is shown in the second column the table below.

| | | Total number of | | MASCOT | |
|---|---|---|---|---|---|
| | Number of | peptides identified | | search score* | |
| | theoretical | Underiva- | Deriva- | Underiva- | Deriva- |
| Protein | peptides⁺ | tised | tised | tised | tised |
| BSA | 144 | 14 (10%) | 41 (28%) | 132 | 126 |
| β-casein | 27 | 4 (15%) | 13 (48%) | no match | 123 |
| ADH | 60 | 7 (12%) | 18 (30%) | 77 | 111 |

⁺The number of theoretical peptides for each protein was generated assuming one missed cleavage and disregarding di- and mono-amino acids generated.
*Score is −10*Log(P), where P is the probability that the observed match is a random event. Protein scores greater than 63 are significant (p < 0.05).

Derivatisation of peptides with trityl groups of the invention thus improves detection, as a significantly larger number of peptides was detected for each of the three proteins when derivatisation was used. Furthermore, protein identification by mass fingerprinting can be improved.

Taking β-casein as an example, the number of detectable fragments more than tripled, and the derivatised spectrum allowed a MASCOT-based identification which was not previously possible.

### Example 8

### BSA Fragmentation and Mass Spectrometry

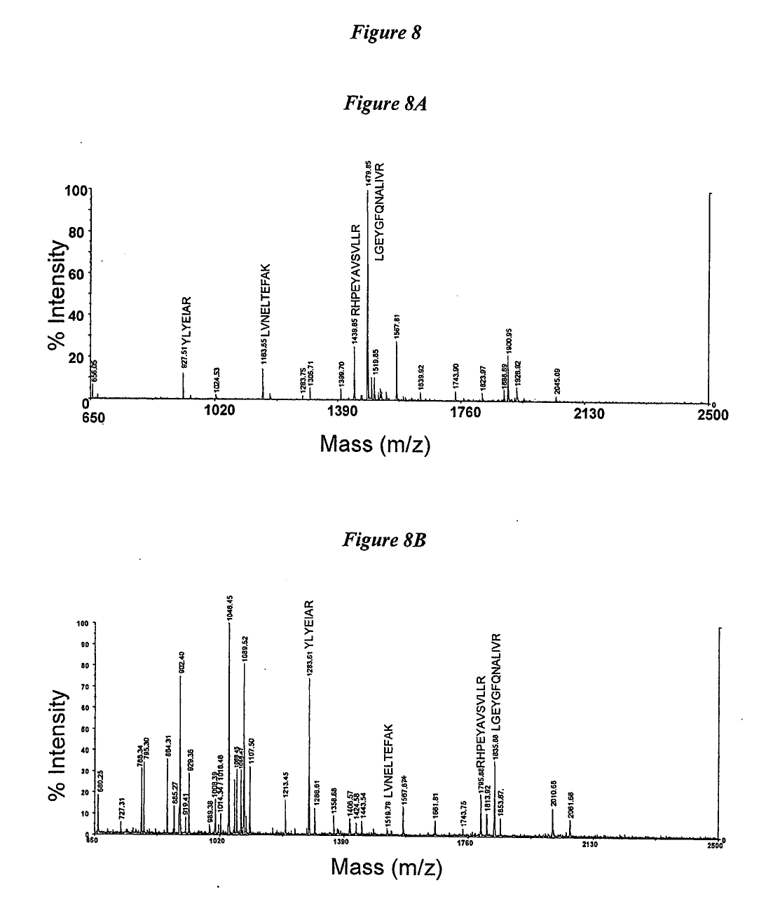

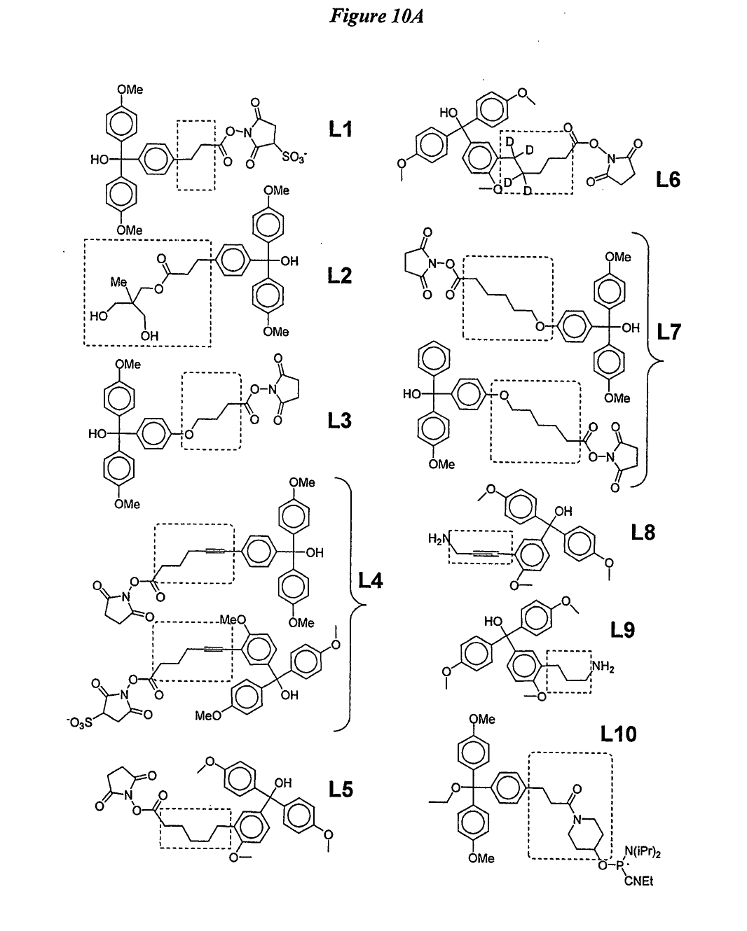

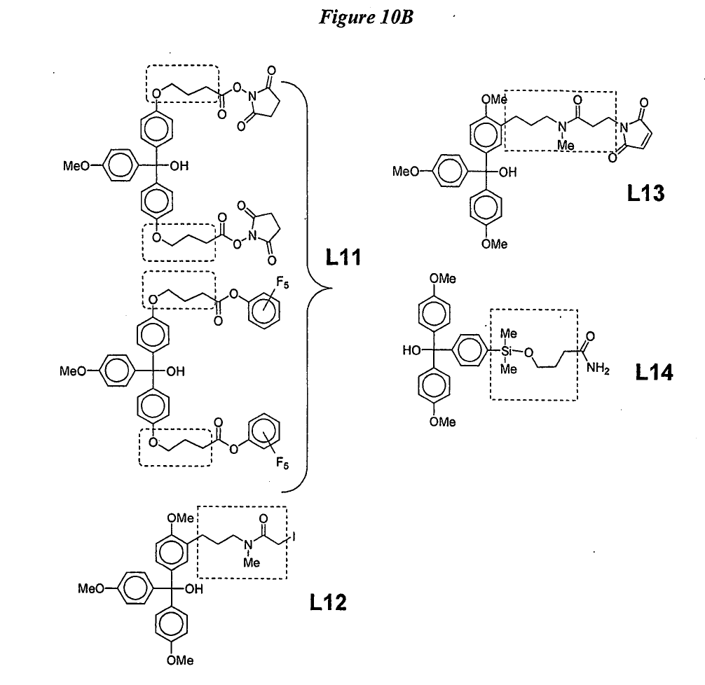

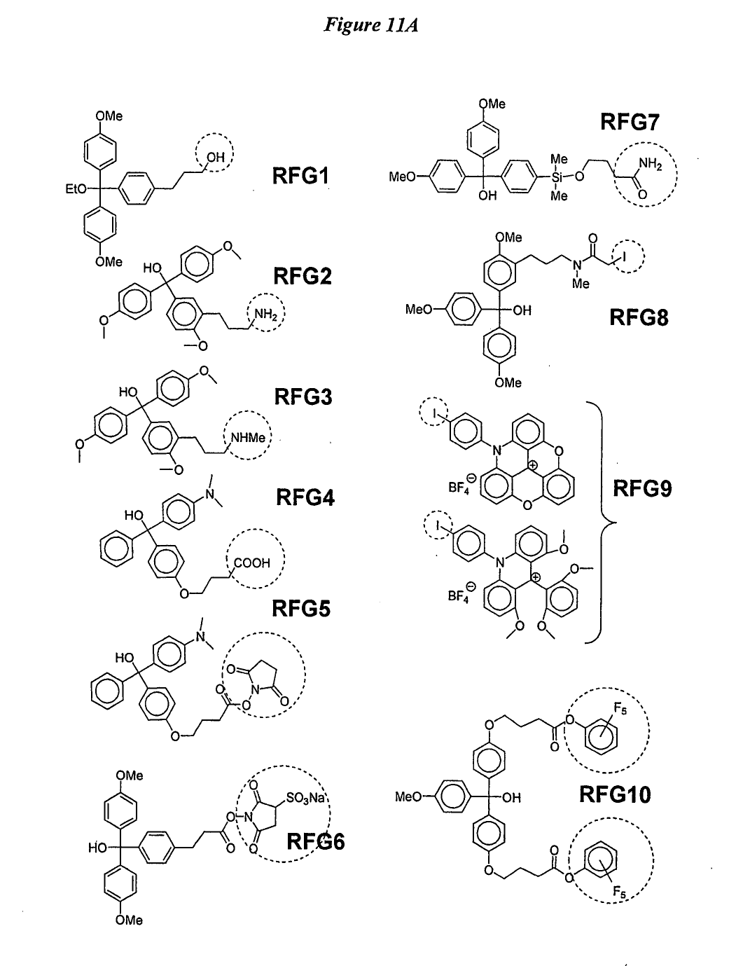

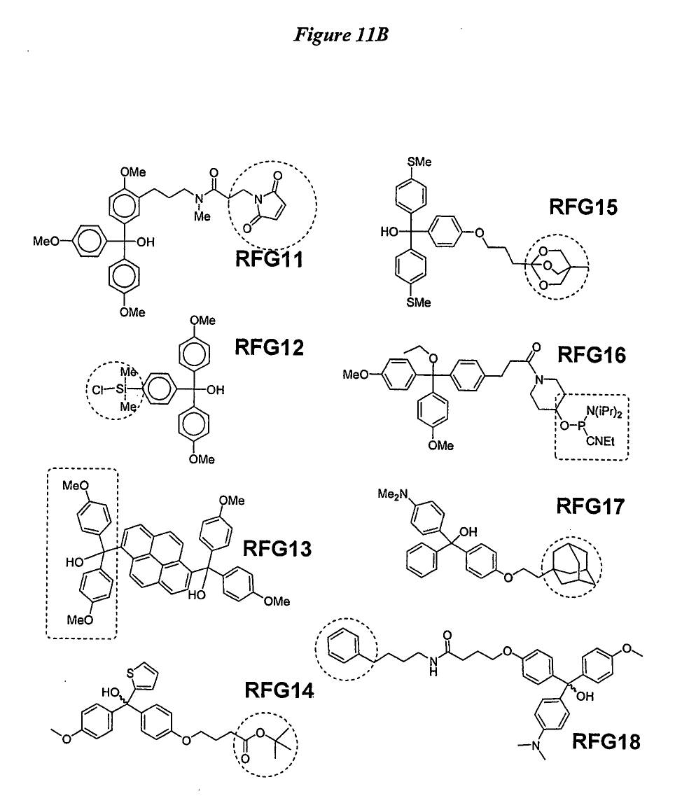

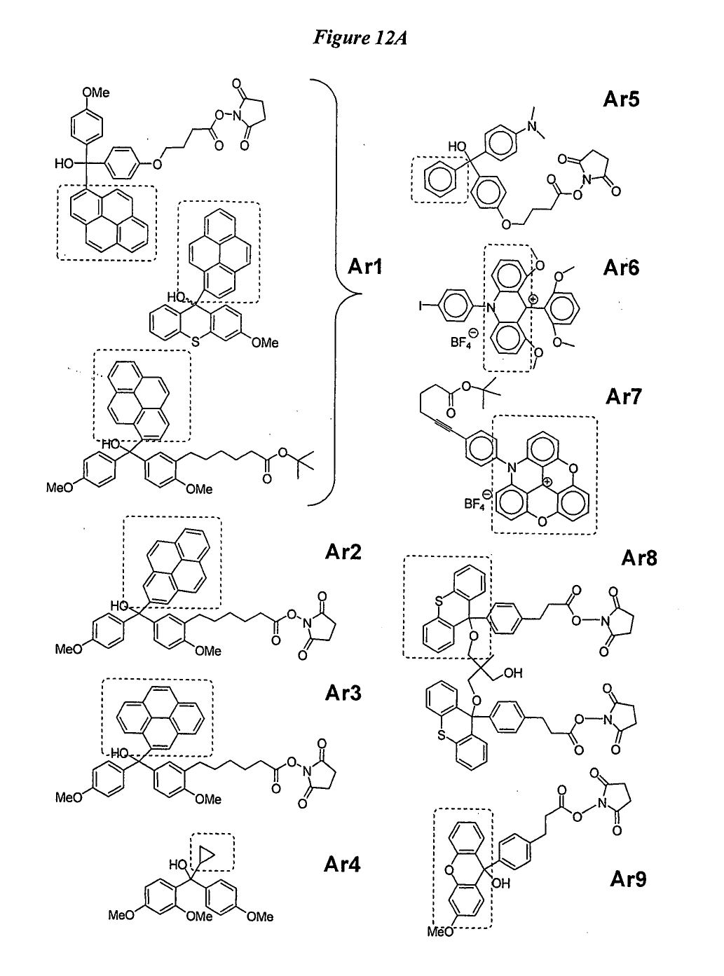

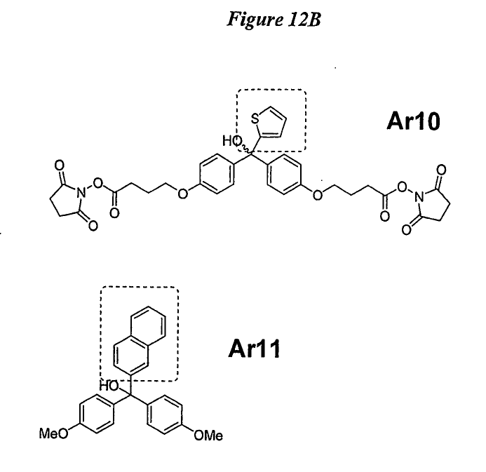

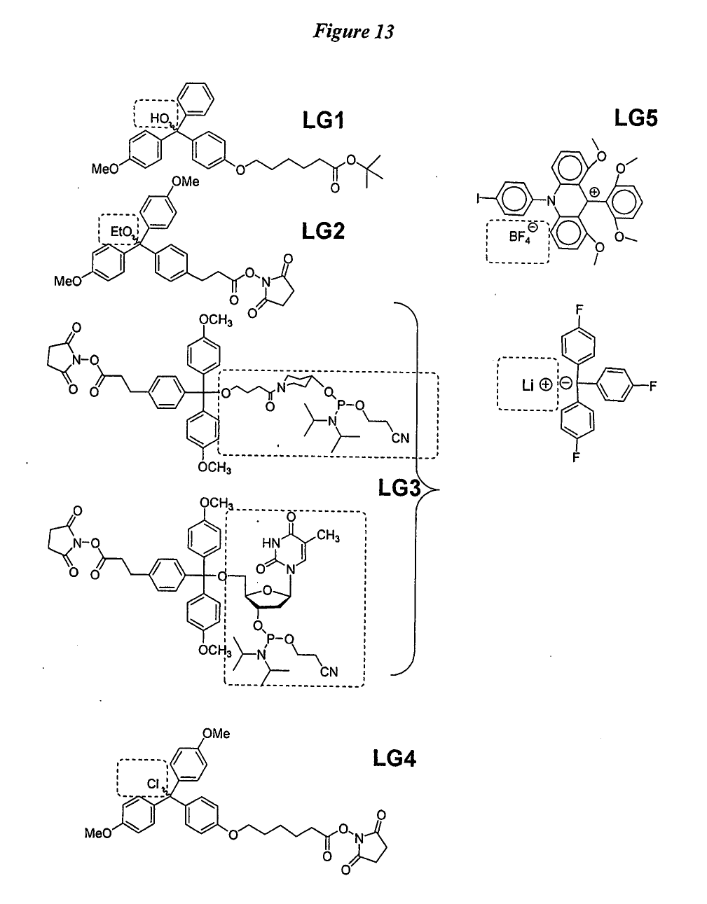

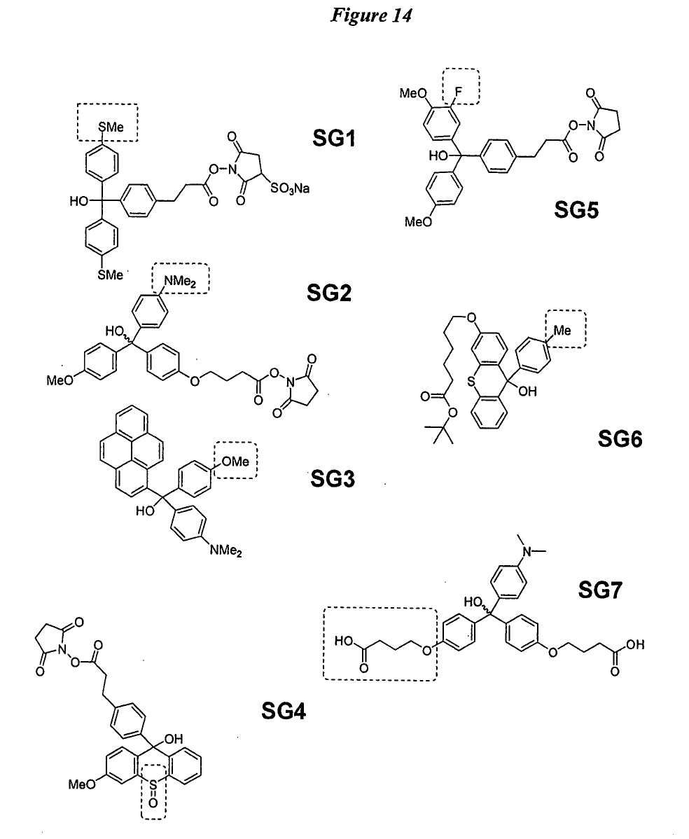

Bovine serum albumin (BSA) was digested with trypsin and analysed by MALDI-TOF. The resulting spectrum is shown in FIG. 8A. The experiment was repeated, but the peptide mixture was labelled with a dimethoxytrityl label after trypsin digestion. The spectrum in FIG. 8B shows the dramatic increase in visible ions due to the trityl label. Four specific peptides have been highlighted in both spectra.

### Example 9

### Mass Spectrometry of Amines

A solution of NHS-activated 4,4′-dimethoxy-4″-carboxyethyl trityl (01M) in THF/dioxane (1:1) was mixed with a solution (0.5-1M) of an amine or of a mixture of amines (for example, propyl amine, butyl amine, pentyl amine, hexyl amine and phenethyl amine), typically 10 ml of a solution of an activated trityl with 5 ml of an amine solution. The mixtures were purified on prep-TLC (2 mm-thick glass plates with UV254 indicator, Analtech/Aldrich-Sigma), typically in chloroform with 0.5% triethylamine. The areas containing the desired products were scratched off the plate, and the conjugates or the mixtures thereof were eluted using same solvent with 2-5% MeOH, filtered through a layer of glass wool, evaporated and dried. FIG. 9 shows a spectrum obtained in this way.

It will be understood that the invention is described above by way of example only and modifications may be made whilst remaining within the scope and spirit of the invention.

1. A method of forming an ion of formula (I): 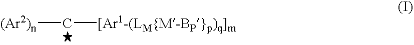
comprising the steps of:
(i) reacting a compound of the formula (IIa): 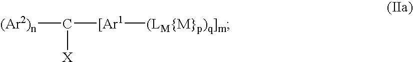
with a biopolymer, B_{P}, having at least one group capable of reacting with M to form a covalent linkage, to provide a biopolymer derivative of the formula (IIIa): 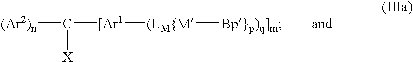
(ii) cleaving the C—X bond between X and the α-carbon atom of the derivative of formula (IIIa) to form the ion of formula (I);
where:
C [symbol] is a carbon atom bearing a single positive charge or a single negative charge;
X is a group capable of being cleaved from the α-carbon atom to form an ion of formula (I);
M is independently a group capable of reacting with B_{P} to form the covalent linkage;
B_{P}′ is independently the biopolymer residue of B_{P} produced on formation of the covalent linkage;
M′ is independently the residue of M produced on formation of the covalent linkage;
Ar¹ is independently an aromatic group or an aromatic group substituted with one or more A;
Ar² is independently an aromatic group or an aromatic group substituted with one or more A;
optionally wherein (a) two or three of the groups Ar¹ and Ar² are linked together by one or more L⁵, where L⁵ is independently a single bond or a linker atom or group; and/or (b) two or three of the groups Ar¹ and Ar² together form, an aromatic group or an aromatic group substituted with one or more A;
A is independently a substituent;
L_{M} is independently a single bond or a linker atom or group;
n=0, 1 or 2 and m=1, 2, or 3, provided the sum of n+m=3;
p independently=1 or more; and
q independently=1 or more.

2. A method of forming an ion of formula (I), comprising the steps of:
(i) reacting a compound of the formula (IIb): 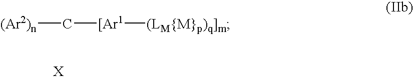
with a biopolymer, B_{P}, having at least one group capable of reacting with M to form a covalent linkage, to provide a biopolymer derivative of the formula (IIIb): 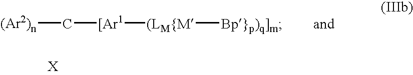
dissociating X [symbol] from the derivative of formula (IIIb), to form the ion of formula (I);
where:
X [symbol] is a counter-ion to C [symbol] ;
and C [symbol] M, B_{P}′, M′, Ar¹, Ar², L_{M}, n, m, p and q are as defined in claim 1.

3. A biopolymer derivative of the formula (IIIa).

4. A biopolymer derivative of the formula (IIIb).

5. An ion of formula (I).

6. A compound of the formula (IIa).

7. A compound of the formula (IIb).

8. A method of forming an ion of formula (I) comprising the steps of:
(i) reacting a solid support of formula (IVai), (IVaii), or (IVaiii): 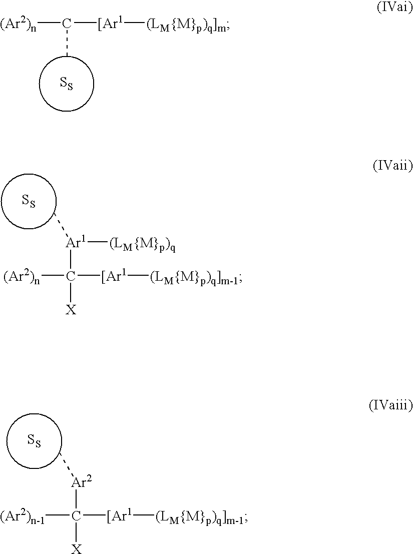
with a biopolymer, B_{P}, having at least one group capable of reacting with M to form a covalent linkage, to provide a modified solid support of the formula (Vai), (Vaii), or (Vaiii), respectively: 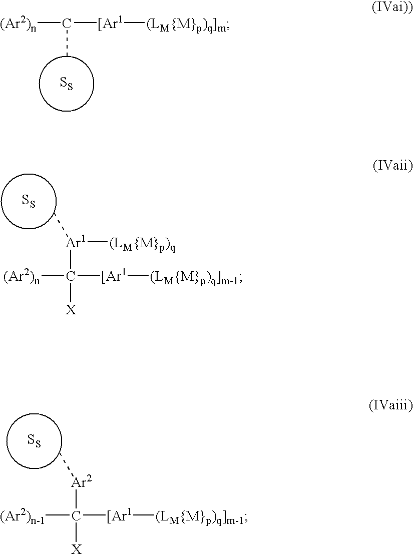
and either:
(iia) for modified solid supports of formula (Vai) cleaving the C—S_{S} bond between the α-carbon atom of the modified solid support of formula (Vai) and the solid support S_{S} to form the ion of formula (I);
(iib) for modified solid supports of formula (Vaii), either simultaneously or sequentially, cleaving the C—X bond between X and the α-carbon atom and cleaving the SS—Ar1 bond between the solid support and the Ar1 group to form the ion of formula (I); or
(iic) for modified solid supports of formula (Vaiii), either simultaneously or sequentially, cleaving the C—X bond between X and the α-carbon atom and cleaving the SS—Ar2 bond between the solid support and the Ar2 group to form the ion of formula (I);
where:
X, Ar¹, Ar², B_{P}′, L_{M}, M, M′, n, m, p and q are as defined in claim 1;
S_{S} is a solid support;
C—S_{S} comprises a cleavable bond between C and S_{S};
S_{S}—Ar¹ comprises a cleavable bond between Ar¹ and S_{S}; and
S_{S}—Ar² comprises a cleavable bond between Ar² and S_{S}.

9. A method of forming an ion of formula (I) comprising the steps of:
(i) reacting a solid support of formula (IVbii) or (IVbiii): 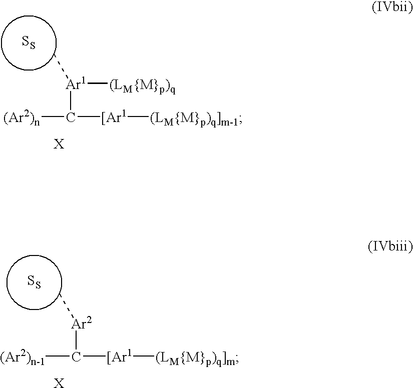
with a biopolymer, B_{P}, having at least one group capable of reacting with M to form a covalent linkage, to provide a modified solid support of the formula (Vbii) or (Vbiii), respectively: 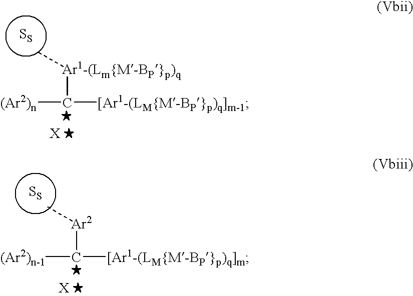
and either:
(iia) for modified solid supports of formula (Vbii), either simultaneously or sequentially, dissociating X [symbol] from the derivative of formula (Vbii) and cleaving the S_{S}—Ar¹ bond between the solid support and the Ar¹ group to form an ion of formula (I); or
(iib) for modified solid supports of formula (Vbiii), either simultaneously or sequentially, dissociating X [symbol] from the derivative of formula (Vbiii) and cleaving the S_{S}—Ar² bond between the solid support and the Ar² group to form an ion of formula (I);
where: X [symbol] Ar¹, Ar², B_{P}′, L_{M}, M, M′, n, m, p, q, S_{S}, C—S_{S}, S_{S}—Ar¹ and S_{S}—Ar² are as defined in claim 8.

10. A method of forming an ion of formula (I) comprising the steps of:
(i) reacting a solid support of formula (IVaiv) or (IVbiv): 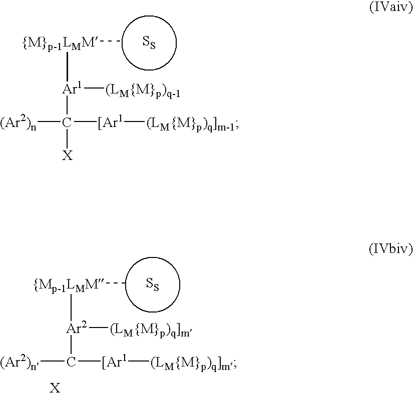
with a biopolymer, B_{P}, having at least one group capable of reacting with M to form a covalent linkage, to provide a modified solid support of the formula (Vaiv) or (Vbiv), respectively: 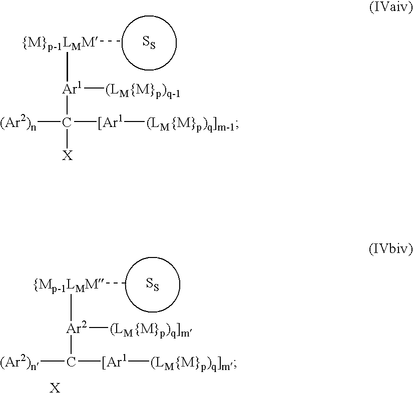
and either:
(iia) for modified solid supports of formula (Vaiv), cleaving the C—X bond between X and the α-carbon atom to form the ion of formula (I); or
(iib) for modified solid supports of formula (Vbiv), dissociating X [symbol] from the derivative of formula (Vbiv) to form the ion of formula (I);
where:
X, X [symbol] Ar¹, Ar², B_{P}′, L_{M}, M, M′, p, q, n, m, and S_{S} are as defined in claims 8 and 9;
m″---S_{S} comprises a bond between M″ and S_{S}; and
M″ is the same as M except that S_{S} is bound to a portion of M which does not form part of M′.

11. A solid support of the formula (IVai), (IVaii), (IVaiii), (IVaiv), (IVbii), (IVbiii) or (IVbiv).

12. A modified solid support of the formula (Vai), (Vaii), (Vaiii), (Vaiv), (Vbii), (Vbiii) or (Vbiv).

13. A method of any of claims 8-10 or a product of claim 11 or 12 wherein the biopolymer is a synthetic biopolymer.

14. A method or product of claim 13 wherein the synthetic biopolymer is an oligonucleotide, a peptide or a carbohydrate.

15. A method for analysing a biopolymer, B_{P}, comprising the steps of:
(i) reacting the biopolymer B_{P} with a compound of formula (IIa) or (IIb) or a solid support of formula (IVai), (IVaii), (IVaiii), (IVaiv), (IVbii), (IVbiii) or (IVbiv);
(ii) providing an ion of formula (I); and
(iii) analysing the ion of formula (I) by mass spectrometry.

16. In a method for analysing a biopolymer, B_{P}, the improvement consisting of: (i) reacting a biopolymer, B_{P} with a compound of formula (IIa) or (IIb) or a solid support of formula (IVai), (IVaii), (IVaiii), (IVaiv), (IVbii), (IVbiii) or (IVbiv); (ii) providing an ion of formula (I); and (iii) analysing the ion by mass spectrometry.

17. A method of claim 15 or claim 16 wherein the analysis by mass spectrometry is carried out in a spectrometer which is suitable for MALDI-TOF spectrometry.

18. A method of any of claims 1, 2, 8-10 or 13-17 or a product of any of claims 3-7, 11 or 12, wherein C [symbol] bears a single positive charge, such that the ions of formulae (I), (IIb) and (IIIb) have the structures:

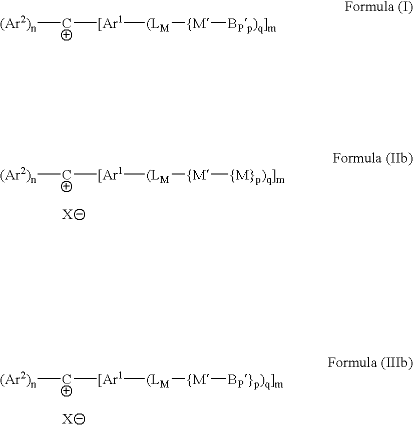

19. A method of any of claims 1, 2, 8-10 or 13-18 or a product of any of claims 3-7, 11, 12 or 18 wherein n=2 and m=1.

20. A method of any of claims 1, 2, 8-10 or 13-19 or a product of any of claims 3-7, 11, 12, 18 or 19 wherein p=1, 2 or 3.

21. A method of any of claims 1, 2, 8-10 or 13-20 or a product of any of claims 3-7, 11, 12 or 18-20 wherein p=1.

22. A method of any of claims 1, 2, 8-10 or 13-21 or a product of any of claims 3-7, 11, 12 or 18-21 wherein q=1, 2 or 3.

23. A method of any of claims 1, 2, 8-10 or 13-22 or a product of any of claims 3-7, 11, 12 or 18-22 wherein q=1.

24. A method of any of claims 1, 2, 8-10 or 13-23 or a product of any of claims 3-7, 11, 12 or 18-23 wherein n=2, m=1, p=1 and q=1, such that the ion of formula (I) has the structure:

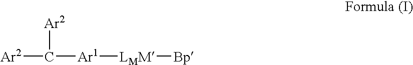

25. A method of any of claims 1, 2, 8-10 or 13-24 or a product of any of claims 3-7, 11, 12 or 18-24 wherein the biopolymer is a polymer found in biological samples.

26. A method or product of claim 25 wherein the biopolymer is a polypeptide, polysaccharide, or polynucleotide.

27. A method or product of claim 26 wherein the biopolymer is a polypeptide.

28. A method or product of any of claims 25-27 wherein the biopolymer does not readily form a molecular ion on illumination of laser light at 340 nm.

29. A method of any of claims 1, 2, 8-10 or 13-28 or a product of any of claims 3-7, 11, 12 or 18-28 wherein the ratio m(B_{P}′)/m(IX) is more than 2, where m(IX) is the mass of the fragment (IX) 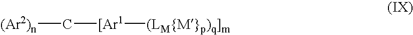
of the cation of formula (I) and m(B_{P}′) is the mass of the biopolymer residue B_{P}′.

30. A method of any of claims 1, 2, 8-10 or 13-29 or a product of any of claims 3-7, 11, 12 or 18-29 wherein M is: —NR₂; —SR; —OR; —B(R)Y; —BY₂; —C(R)₂Y; —C(R)Y₂; —CY₃; —C(=Z)Y; -Z-C(=Z)Y; —C(=Z)R; —C(R)(OH)OR; —C(R)(OR)₂; —S(═O)Y; -Z-S(═O)Y; —S(═O)₂Y; -Z-S(═O)₂Y; —S(═O)₃Y; -Z-S(═O)₃Y; —P(=Z)(ZR)Y; —P(=Z)Y₂; -Z-P(=Z)(ZR)Y; -Z-P(=Z)Y₂; —P(=Z)(R)Y; -Z-P(=Z)(R)Y; or —N═C(=Z), where Y is independently a leaving group, Z is independently O, S or N(R) and R is independently H, C₁₋₈hydrocarbyl or C₁₋₈hydrocarbyl substituted with one or more A.

31. A method of any of claims 1, 2, 8-10 or 13-29 or a product of any of claims 3-7, 11, 12 or 18-29 wherein M is: —N(R)—; —S—; —O—; —B(Y)—; —C(R)(Y)—; —CY₂—; —C(═O)—; —C(OH)(OR)—; or —C(OR)₂—, where Y is independently a leaving group and R is independently H, C₁₋₈hydrocarbyl or C₁₋₈hydrocarbyl substituted with one or more A.

32. A method of any of claims 1, 2, 8-10 or 13-29 or a product of any of claims 3-7, 11, 12 or 18-29 wherein M is: 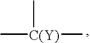
where Y is a leaving group.

33. A method of any of claims 1, 2, 8-10 or 13-29 or a product of any of claims 3-7, 11, 12 or 18-29 wherein the covalent linkage is selected from those produced through the reaction of one the following groups: —CO—NH—; biotin-(strept)avidin; 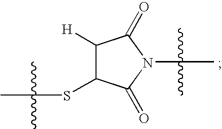
or —NH—CS—NH—.

34. A method of any of claims 1, 2, 8-10 or 13-33 or a product of any of claims 3-7, 11, 12 or 18-33 wherein L_{M} is O or S.

35. A method of any of claims 1, 2, 8-10 or 13-33 or a product of any of claims 3-7, 11, 12 or 18-33 wherein L_{M} is -E^{M}-, -(D^{M})ₜ-, -(E^{M}-D^{M})ₜ-, -(D^{M}-E^{M})ₜ-, -E^{M}(D^{M}-E^{M})ₜ- or -D^{M}-(E^{M}-D^{M})ₜ- (in the orientation Ar¹-(L_{M}{M}ₚ)_{q} or Ar¹-(L_{M}{M′}ₚ)_{q}, as appropriate),
where:
a sufficient number of linking covalent bonds, in addition to the covalent bonds at the chain termini shown, are provided on groups E^{M} and D^{M} for linking the p instances of M (or M′) groups;
D^{M} is independently C₁₋₈hydrocarbylene or C₁₋₈hydrocarbylene substituted with one or more A;
E^{M} (in the orientation Ar¹-(L_{M}{M}ₚ)_{q} or Ar¹-(L_{M}{M′}ₚ)_{q}, as appropriate) is independently -Z^{M}-, —C(=Z^{M})-, -Z^{M}C(=Z^{M})-, C(=Z^{M})Z^{M}- , -Z^{M}C(=Z^{M})Z^{M}-, —S(═O)—, -Z^{M}S(═O)—, —S(═O)Z^{M}-, -Z^{M}S(═O)Z^{M}-, —S(═O)₂—, -Z^{M}S(═O)₂—, —S(═O)₂Z^{M}-, -Z^{M}S(═O)₂Z^{M}-, where Z^{M} is independently O, S or N(R^{M}) and where R^{M} is independently H, C₁₋₈hydrocarbyl (e.g. C₁₋₈alkyl) or C₁₋₈hydrocarbyl substituted with one or more A; and
t=1 or more.

36. A method of any of claims 1, 8, 10 or 13-35 or a product of any of claims 3, 6, 11, 12 or 18-35 wherein the group X is halogen, hydroxy, C₁₋₈hydrocarbyloxy, C₁₋₈hydrocarbyloxy substituted with one or more A, C₁₋₈heterohydrocarbyloxy, C₁₋₈heterohydrocarbyloxy substituted with one or more A, mesyl, tosyl, pentafluorophenyl, —O-succinimidyl —S-succinimidyl, or phenyloxy substituted with one or more A.

37. A method of any of claims 1, 2, 8-10 or 13-36 or a product of any of claims 3-7, 11, 12 or 18-36 wherein Ar² is independently cyclopropyl, cyclopropyl substituted with one or more A, aryl, aryl substituted with one or more A, heteroaryl, or heteroaryl substituted with one or more A.

38. A method of any of claims 1, 2, 8-10 or 13-37 or a product of any of claims 3-7, 11, 12 or 18-37 wherein Ar² is

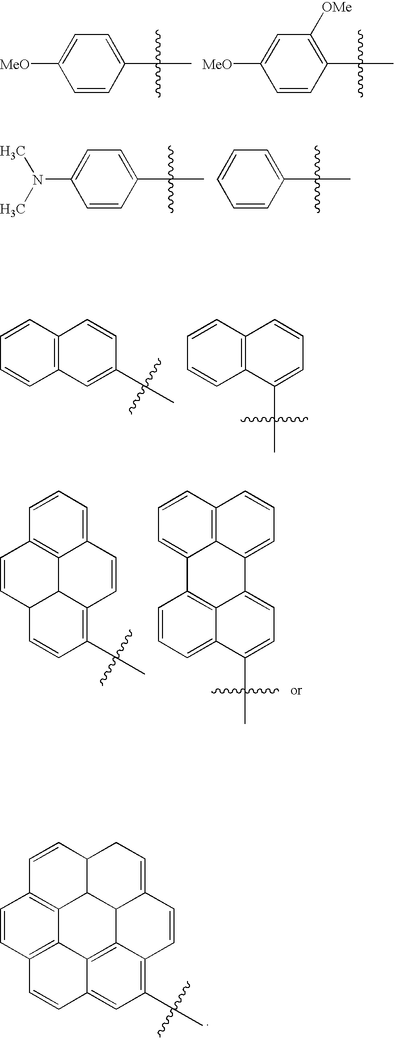

39. A method of any of claims 1, 2, 8-10 or 13-38 or a product of any of claims 3-7, 11, 12 or 18-38 wherein Ar¹ is independently cyclopropylene, cyclopropylene substituted with one or more A, arylene, arylene substituted with one or more A, heteroarylene, or heteroarylene substituted with one or more A.

40. A method of any of claims 1, 2, 8-10 or 13-39 or a product of any of claims 3-7, 11, 12 or 18-39 wherein Ar¹ is

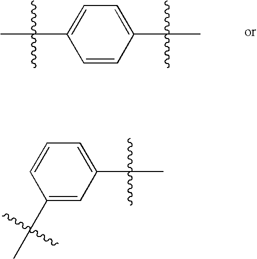

41. A method of any of claims 1, 2, 8-10 or 13-40 or a product of any of claims 3-7, 11, 12 or 18-40 wherein L⁵ is O or S.

42. A method of any of claims 1, 2, 8-10 or 13-40 or a product of any of claims 3-7, 11, 12 or 18-40 wherein L⁵ is -E⁵-, -(D⁵)_{t′}-, -(E⁵-D⁵)_{t′}-, -(D⁵-E⁵)_{t′}-, -E⁵-(D⁵-E⁵)_{t′}- or -D⁵-(E⁵-D⁵)_{t′}-
where:
D⁵ is independently C₁₋₈hydrocarbylene or C₁₋₈hydrocarbylene substituted with one or more A;
E⁵ is independently -Z⁵-, —C(=Z⁵)-, -Z⁵C(=Z⁵)-, —C(=Z⁵)Z⁵-, -Z⁵C(=Z⁵)Z⁵-, —S(═O)—, -Z⁵S(═O)—, —S(═O)Z⁵-, -Z⁵S(═O)Z⁵-, —S(═O)₂—, -Z⁵S(═O)₂—, —S(═O)₂Z⁵-, -Z⁵S(═O)₂Z⁵-, where Z⁵ is independently O, S or N(R⁵) and where R⁵ is independently H, C₁₋₈hydrocarbyl or C₁₋₈hydrocarbyl substituted with one or more A; and
t′ or more.
